(19) United States
(12) Patent Application Publication
(10) Pub. No.: US 2026/0315505 A1
(43) Pub. Date: Oct. 15, 2026

(54) COMBINED INHIBITION OF TYROSINE PHOSPHATASES TCPTP AND PTP1B AND PD-1 BLOCKAGE IMMUNOTHERAPY IN THE TREATMENT OF CANCER

(71) Applicant: Kanyr Pharma Inc., Boucherville (CA)

(72) Inventors: Michel L. Tremblay, 4250 Vendome (CA); Daniel BOUTHILLIER, Boucherville (CA)

(21) Appl. No.: 19/481,387

(22) PCT Filed: May 19, 2023
(86) PCT No.: PCT/CA2023/050693

§ 371 Date: Nov. 4, 2025

Related U.S. Application Data
(60) Provisional application No. 63/343,662, filed on May 19, 2022.

(51) Int. Cl. A61K 31/675; A61K 31/662; A61K 31/665; A61K 39/00; A61K 39/395; A61P 35/00

(52) U.S. Cl. CPC A61K 31/675; A61K 31/662; A61K 31/665; A61K 39/3955; A61P 35/00; A61K 2039/505

(57) ABSTRACT

The present document describes methods for the treatment of cancer comprising administering to a subject in need thereof a therapeutically effective amount of a compound of structural Formula (I) or Formula (II), or pharmaceutically acceptable salts thereof, and stereoisomers thereof, in combination with an immune checkpoint inhibitor, for the treatment of cancer.

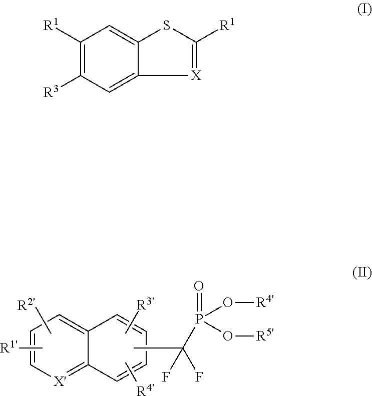

### CROSS-REFERENCE TO RELATED APPLICATIONS

This application claims priority of U.S. Provisional Patent Application No. 63/343,662 filed on May 19, 2022, the specification of which is hereby incorporated by reference in its entirety.

### BACKGROUND

### (a) Field

The subject matter disclosed generally relates to a method for the treatment of cancer, and more specifically to a method for the treatment of cancer comprising administering to a subject in need thereof a therapeutically effective amount of a compound of structural Formula I, of structural Formula II, or pharmaceutically acceptable salts thereof, and stereoisomers thereof, or combinations thereof, in combination with an immune checkpoint inhibitor.

### (b) Related Prior Art

CD8 T cells are essential immune system components in mounting antitumoral responses [1]. There is a positive correlation between the presence of CD8+ tumor infiltrating lymphocytes (TILs) and successful clinical outcomes in several types of cancer [8-10]. Moreover, cancer regression is observed after clonal CD8+ cell repopulation in melanoma patients [11], setting the grounds for exploring CD8+ cell based therapies. Indeed, redirection and/or reactivation of immune responses against cancer has been a remarkable source for novel therapies as immune checkpoint blockade [12, 13]. Immune checkpoint inhibitors (ICI) anti-PD-1 directed antibodies are extensively used for clinical trial and have been approved by the FDA for almost half of malignancies. They nevertheless still have a low estimated level of responsiveness of 12.5% and failed to increase life span or progression free survival significantly [14]. Checkpoint inhibitors work by preventing/reversing a dysfunctional state of CD8 T cells known as exhaustion [15, 16]. Exhausted CD8 T cells are the result of chronic activation and is characterized by the expression of several inhibitory receptors including CTLA-4, PD-1, Tim-3, TIGIT, SLAMF6 [17]. The limited success of ICI emphasizes the multifactorial nature of CD8 T cell dysfunction and encourage the search for novel strategies to prevent it. Indeed most of know checkpoint receptors act by modulating tyrosine phosphorylation through the recruitment of SHP-1 and SHP-2 phosphatases [17]. Hence the role of other regulatory phosphatases needs to be explored.

TCPTP (PTPN2) and PTP1B (PTPN1) are two highly homologous nonreceptor protein tyrosine phosphatases (NRPTP) with preferential activity on targets of the Jak/STAT pathway [2]. While TCPTP role as immune regulator is known [3, 4, 18], recent studies have underscored the potential of this phosphatase as an immunotherapeutic target [7, 19-21]. PTP1B on the other hand, although less studied has been linked to an immune regulatory role [7, 22]. Whilst substrate trapping mutant studies propose a mainly non-redundant role between both genes, PTP1B targeting Jak2, Tyk 2 and STAT6 [23, 24] while TCPTP preferentially dephosphorylating Jak1, Jak3, STAT 1 and STAT5 [25-27], attempts to block the genetic expression of both enzymes highlight a potential cooperative overlapping role in the phosphorylation of STAT1 [6]. Embryonic lethality of double deletion in mice strongly suggest this overlapping role, as single mutant animals reach birth at mendelian rates [28, 29].

To explore the potential synergy of both enzymes in CD8+ T cells, CD8+ cells from mice with conditionally targeted deletions of TCPTP and PTP1 b in mature CD8+ T cells are herein analyzed.

### SUMMARY

According to an embodiment, there is provided a method for the treatment of cancer comprising administering to a subject in need thereof a therapeutically effective amount of a compound of structural Formula II, or pharmaceutically acceptable salts thereof, and stereoisomers thereof:

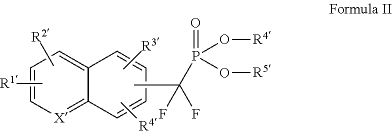

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group,
R^{4′} and R^{5′} are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
   (i) —(C═O)OR⁷,
   (ii) —(C═O)NHR⁷,
   (iii) —(C═O)N(R⁷)₂,
   (iv) —(C═O)NH₂,
   (v) —OR⁷,
   (vi) —O(C═O)R⁷,
   (vii) —O(C═O)OR⁷,
   (viii) —O(C═O)NHR⁷,
   (ix) —O(C═O)N(R⁷)₂,
   (x) —O(C═O)NH₂,
   (xi) —SO₂NH₂,
   (xii) —SOₓCH₃,
   (viii) —S(C═O)R⁷ and
   (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl; and
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R⁷ is selected from the group consisting of C₁₋₆alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —OOOH, and —OC₁₋₃ alkyl;
R^{a} and R^{b} are each independently hydrogen or C₁₋₄ alkyl optionally substituted with hydroxy or 1-5 fluorines; and
x is 0, 1, or 2;
   in combination with an immune checkpoint inhibitor.

According to another embodiment, there is provided a method for the treatment of cancer comprising administering to a subject in need thereof a therapeutically effective amount of a compound of structural Formula I, or pharmaceutically acceptable salts thereof, and stereoisomers thereof:

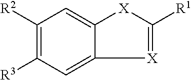

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁ 3 alkyl or C₁₋₃ haloalkyl; and
x is an integer from 0 to 2;
   in combination with an immune checkpoint inhibitor.

According to another embodiment, there is provided a method for the treatment of cancer comprising administering to a subject in need thereof a therapeutically effective amount of a combination of a compound of structural Formula I and of structural Formula II, or pharmaceutically acceptable salts thereof, and stereoisomers thereof:

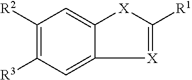

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —OOOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁ 3 alkyl or C₁₋₃ haloalkyl; and
x is an integer from 0 to 2;

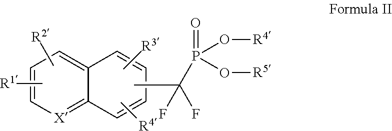

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group,
R^{4′} and R^{5′} are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
   (i) —(C═O)OR⁷,
   (ii) —(C═O)NHR⁷,
   (iii) —(C═O)N(R⁷)₂,
   (iv) —(C═O)NH₂,
   (v) —OR⁷,
   (vi) —O(C═O)R⁷,
   (vii) —O(C═O)OR⁷,
   (viii) —O(C═O)NHR⁷,
   (ix) —O(C═O)N(R⁷)₂,
   (x) —O(C═O)NH₂,
   (xi) —SO₂NH₂,
   (xii) —SOₓCH₃,
   (viii) —S(C═O)R⁷ and
   (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl; and
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R⁷ is selected from the group consisting of C₁₋₆alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —OOOH, and —OC₁₋₃ alkyl;
R^{a} and R^{b} are each independently hydrogen or C₁₋₄ alkyl optionally substituted with hydroxy or 1-5 fluorines; and
x is 0, 1, or 2;
   in combination with an immune checkpoint inhibitor.

According to another embodiment, there is provided the use of a compound of structural Formula II, or pharmaceutically acceptable salts thereof, and stereoisomers thereof, in combination with an immune checkpoint inhibitor, for the treatment of cancer:

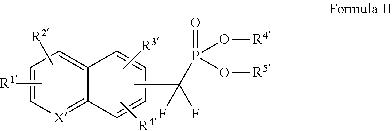

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group,
R^{4′} and R^{5′} are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
   (i) —(C═O)OR⁷,
   (ii) —(C═O)NHR⁷,
   (iii) —(C═O)N(R⁷)₂,
   (iv) —(C═O)NH₂,
   (v) —OR⁷,
   (vi) —O(C═O)R⁷,
   (vii) —O(C═O)OR⁷,
   (viii) —O(C═O)NHR⁷,
   (ix) —O(C═O)N(R⁷)₂,
   (x) —O(C═O)NH₂,
   (xi) —SO₂NH₂,
   (xii) —SOₓCH₃,
   (viii) —S(C═O)R⁷ and
   (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl; and
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R⁷ is selected from the group consisting of C₁₋₆alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃ alkyl;
R^{a} and R^{b} are each independently hydrogen or C₁₋₄ alkyl optionally substituted with hydroxy or 1-5 fluorines; and
x is 0, 1, or 2.

According to another embodiment, there is provided use of a compound of structural Formula I, or pharmaceutically acceptable salts thereof, and stereoisomers thereof in combination with an immune checkpoint inhibitor for the treatment of cancer:

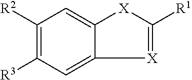

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁₋₃ alkyl or C₁₋₃ haloalkyl; and
x is an integer from 0 to 2.

According to another embodiment, there is provided the use of a combination of a compound of structural Formula I and of structural Formula II, or pharmaceutically acceptable salts thereof, and stereoisomers thereof in combination with an immune checkpoint inhibitor for the treatment of cancer:

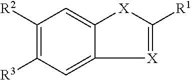

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃ alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁₋₃ alkyl or C₁₋₃ haloalkyl; and
x is an integer from 0 to 2;

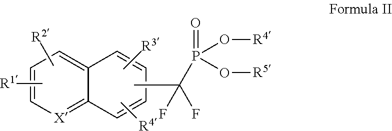

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group, R^{4′} and R^{5′} are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
   (i) —(C═O)OR⁷,
   (ii) —(C═O)NHR⁷,
   (iii) —(C═O)N(R⁷)₂,
   (iv) —(C═O)NH₂,
   (v) —OR⁷,
   (vi) —O(C═O)R⁷,
   (vii) —O(C═O)OR⁷,
   (viii) —O(C═O)NHR⁷,
   (ix) —O(C═O)N(R⁷)₂,
   (x) —O(C═O)NH₂,
   (xi) —SO₂NH₂,
   (xii) —SOₓCH₃,
   (viii) —S(C═O)R⁷ and
   (ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC 1-3 haloalkyl;
or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl; and
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R⁷ is selected from the group consisting of C₁₋₆alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃ alkyl;
R^{a} and R^{b} are each independently hydrogen or C₁₋₄ alkyl optionally substituted with hydroxy or 1-5 fluorines; and
   x is 0, 1, or 2.

The compound of formula II may be of structural Formula IIa, or a pharmaceutically acceptable salts thereof, and stereoisomers thereof:

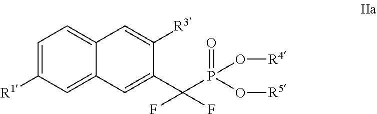

wherein
R^{1′} is selected from the group consisting of (a) C₁₋₃ alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —HC═NOH, (e) —(CH₃)C═NOH, (f) —HC═NOC₁₋₃alkyl optionally substituted with 1-3 halogens, (g) —(CH₃)C═NOC₁₋₃alkyl optionally substituted with 1-3 halogens, (h) —C(═O)OC₁₋₃alkyl optionally substituted with 1-3 halogens, (i) —C(═O)NHR⁶, (j) —CH═CH-Phenyl wherein —CH═CH— is optionally substituted with 1-2 substituents independently selected from halogen and C₁₋₂alkyl optionally substituted with 1-3 F, (k) —CH₂CH₂-Phenyl wherein —CH₂CH₂— is optionally substituted with 1-4 substituents independently selected from halogen and C₁₋₂ alkyl optionally substituted with 1-3 F, (1) Phenyl, (m) -HET-Phenyl, wherein HET is a 5- or 6-membered heteroaromatic ring containing 1-3 heteroatoms selected from O, N and S, (n) —C≡C-Phenyl, (o) —CH₂-Phenyl, and (p) —CN, wherein the —CH₂— group of —CH₂-Phenyl is optionally substituted with 1-2 substituents independently selected from halogen and C₁₋₂ alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{3′} is halogen;
R⁴ and R⁵ are each independently selected from the group consisting of:
(a) hydrogen:
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
   (i) —(C═O)OR⁷,
   (ii) —(C═O)NHR⁷,
   (iii) —(C═O)N(R⁷)₂,
   (iv) —(C═O)NH₂,
   (v) —OR⁷,
   (vi) —O(C═O)R⁷,
   (vii) —O(C═O)OR⁷,
   (viii) —O(C═O)NHR⁷,
   (ix) —O(C═O)N(R⁷)₂,
   (x) —O(C═O)NH₂,
   (xi) —SO₂NH₂,
   (xii) —SOₓCH₃,
   (viii) —S(C═O)R⁷, and
   (xiii) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
   or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
   R^{6′} is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃alkyl optionally substituted with 1-3 halogens, and (v) —OC₁₋₃alkyl optionally substituted with 1-3 halogens;
   R⁷ is selected from the group consisting of C₁₋₆ alkyl optionally substituted with 1-3 substituents independently selected from (i) halogen, (ii) —OC₁₋₃ alkyl, (iii) aryl, and (iv) heteroaryl, wherein the aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃alkyl;
R^{a} and R^{b} are each independently hydrogen or C₁₋₄ alkyl optionally substituted with hydroxy or 1-5 fluorines; and
x is 0, 1, or 2.

The compound may be selected from the following compounds:

| Ex. | Structure |
|---|---|
| 2 | 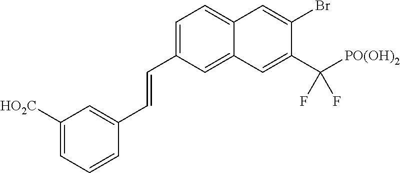 |
| 3 | 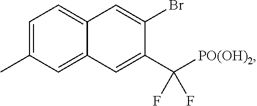 |
| 4 | 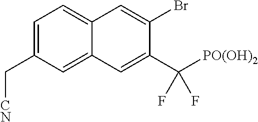 |
| 5 | 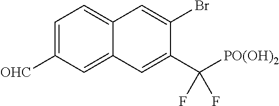 |
| 6 | 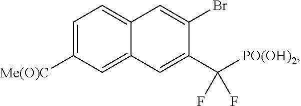 |
| 8a | 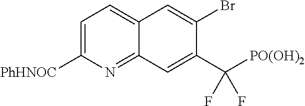 |
| 8b | 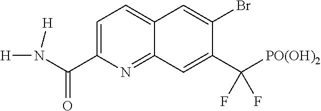 |
| 8c | 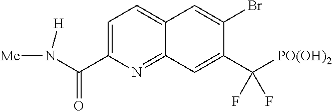 |
| 8d | 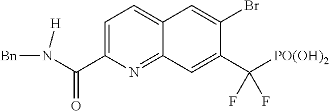 |
| 8e | 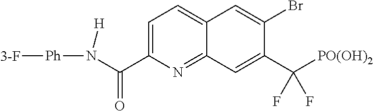 |
| 9a | 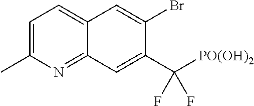 |
| 9c | 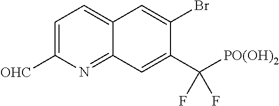 |
| 9d | 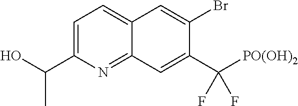 |
| 9e | 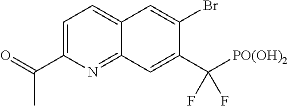 |
| 9f | 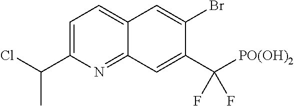 |
| 9g | 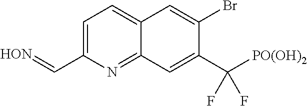 |
| 9h | 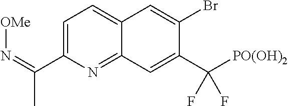 |
| L598 | 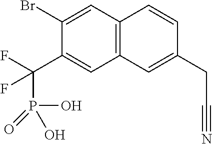 [SMILES: N#CCc1ccc2cc(Br)c(C(F)(F)P(=O)(O)O)cc2c1] |
| 10 | 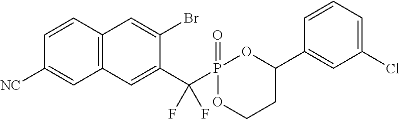 [SMILES: N#Cc1ccc2cc(Br)c(C(F)(F)P3(=O)OCCC(c4cccc(Cl)c4)O3)cc2c1] |
| 11 | 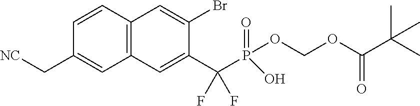 [SMILES: CC(C)(C)C(=O)OCOP(=O)(O)C(F)(F)c1cc2cc(CC#N)ccc2cc1Br] |
| 12 | 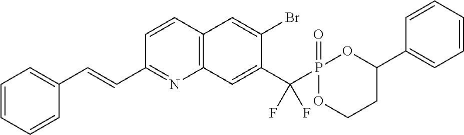 [SMILES: O=P1(C(F)(F)c2cc3nc(/C=C/c4ccccc4)ccc3cc2Br)OCCC(c2ccccc2)O1] |
| 13 | 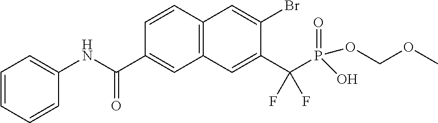 [SMILES: COCOP(=O)(O)C(F)(F)c1cc2cc(C(=O)Nc3ccccc3)ccc2cc1Br] |
| 14 | 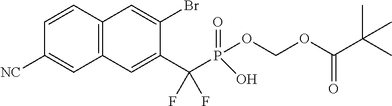 [SMILES: CC(C)(C)C(=O)OCOP(=O)(O)C(F)(F)c1cc2cc(C#N)ccc2cc1Br] |
| 15 | 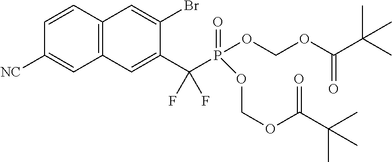 [SMILES: CC(C)(C)C(=O)OCOP(=O)(OCOC(=O)C(C)(C)C)C(F)(F)c1cc2cc(C#N)ccc2cc1Br] |

or a pharmaceutically acceptable salt thereof.

The compound of formula (IIa) may be

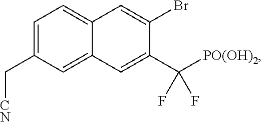

or a pharmaceutically acceptable salt thereof.

The compound of formula (IIa) may be

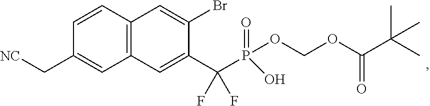 [SMILES: CC(C)(C)C(=O)OCOP(=O)(O)C(F)(F)c1cc2cc(CC#N)ccc2cc1Br]

or a pharmaceutically acceptable salt thereof.

The compound of Formula I may be of structural Formula Ia, or a pharmaceutically acceptable salts thereof, and stereoisomers thereof:

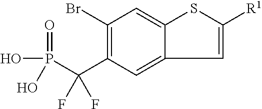

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compound of formula I may be of structural Formula Ib, or a pharmaceutically acceptable salts thereof, and stereoisomers thereof:

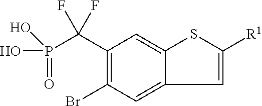

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compound may be a compound selected from the following compounds:

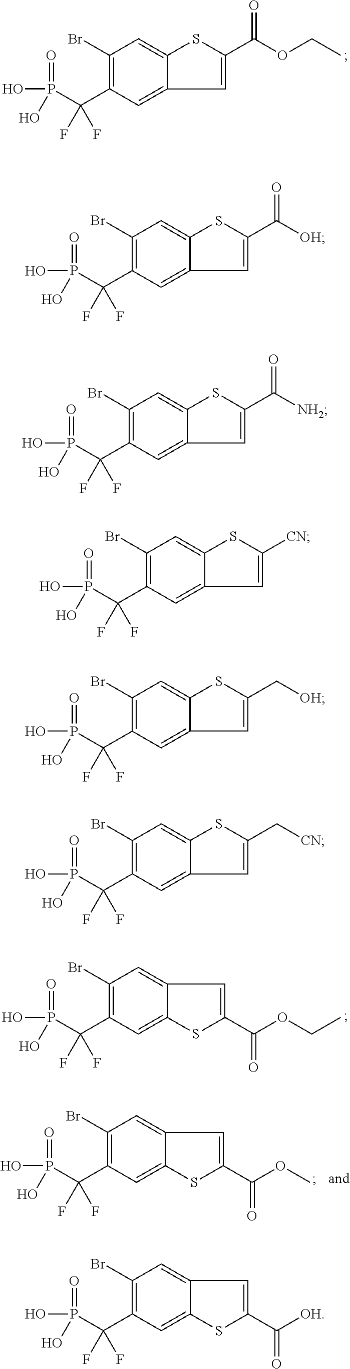

The immune checkpoint inhibitor may be an anti-PD-1 antibody, an anti-PD-L11 antibody, an anti-CTLA-4 antibody, or molecular inhibitors of these receptors.

The anti-PD-1 antibody may be selected from the group consisting of nivolumab antibody, pembrolizumab antibody, pidilizumab antibody, cemiplimab antibody, dostarlimab antibody, JTX-4014 antibody, Spartalizumab antibody, Camrelizumab antibody, Sintilimab antibody, Tislelizumab antibody, Toripalimab antibody, INCMGA00012 antibody, AMP-224 antibody, AMP-514 antibody, RMP1-14 antibody or combinations thereof.

The anti-PDL-1 antibody may be selected from the group consisting of B7-H1 antibody, BMS-936559 antibody, MPDL3280A (atezolizumab) antibody, MEDI-4736 antibody, MSB0010718C antibody, 10F-9G2 antibody, avelumab antibody, durvalumab antibody, or combinations thereof.

The anti-CTLA-4 antibody may be selected from the group consisting of ipilimumab or tremelimumab or combinations thereof.

The administration of the compound of structural Formula I or structural Formula II may be an intratumoral administration.

The administration of the compound of structural Formula I or structural Formula II is an oral administration.

The administration of the immune checkpoint inhibitor may be a systemic administration, and intratumoral administration, or a combination thereof.

The administration of the compound of structural Formula I or structural Formula II may be an intratumoral administration and administration of the immune checkpoint inhibitor may be a systemic administration, and intratumoral administration, or a combination thereof, or administration of the compound of structural Formula I may be an intratumoral administration and administration of the immune checkpoint inhibitor may be a systemic administration, an intratumoral administration, or a combination thereof, or administration of the compound of structural Formula I may be an oral administration and administration of the immune checkpoint inhibitor may be a systemic administration, an intratumoral administration, or a combination thereof.

The compound of structural Formula I or structural Formula II may be administered first, and the immune checkpoint inhibitor is administered second.

The cancer may be a solid tumor, a prostate cancer, a breast cancer, a brain cancer, a glioma, a lung cancer, a salivary cancer, a stomach cancer, a thymic epithelial cancer, a thyroid cancer, an ovarian cancer, a multiple myeloma, a leukemia, a melanoma, a lymphoma, a gastric cancer, a kidney cancer, a pancreatic cancer, a bladder cancer, a colon cancer and a liver cancer.

The method or the use of the present invention may further comprise monitoring the activity of the compound of structural Formula I, structural Formula II or both structural Formula I and structural Formula II.

The following terms are defined below.

The term “antibody”, which is also referred to in the art as “immunoglobulin” (Ig), as used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (C_{L}) domain, while the heavy chain folds into a variable (V_{H}) and multiple constant (e.g., C_{H}1, C_{H}2, C_{H}3) domains. Interaction of the heavy and light chain variable domains (V_{H} and V_{L}) results in the formation of an antigen binding region (Fv). Each domain has a well-established structure familiar to those of skill in the art.

The present invention further encompasses “monoclonal antibodies” (mAb). Monoclonal antibodies are antibodies that are made by identical immune cells which are all clones belonging to a unique parent cell. Monoclonal antibodies can have monovalent affinity, in that they bind to the same epitope (the part of an antigen that is recognized by the antibody).

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A illustrates that CD8 T cells activation and differentiation is increased in naïve mice with reduced expression of TCPTP and/or PTP1B. Ex vivo analysis of peripheral T cells population of naïve 6 to 8 weeks old mice with conditional deletions of Ptpn1 and/or Ptpn2 in CD8 T cells by multiparametric flow cytometric analysis of T cells from spleen and lymph nodes. The panels show the CD4 vs CD8 cells from different genotypes. Events shown correspond to single, viable and lineage negative (Lin−) gates.

FIG. 1B shows that statistical representation of T cell subtypes from different experiments organized by genotype.

FIG. 1C shows histogram representation of T cell subtypes from different experiments organized by genotype.

FIG. 1D shows the quantification of CD8 low populations in spleen of different genotypes.

FIG. 1E shows dot plots of CD8 T cell differentiation determined by CD62L and CD44 expression. FIG. 1F shows the statistical representation of CD8 T cell differentiation determined by CD62L and CD44 expression.

FIG. 1G a histogram representation of differentiation markers EOMES, KLRG1 and CD127. (CRE n=4, 1BKO n=2, TCKO n=4, TCKO/1BHet n=2, dKO n=4) (Bars represent S.E.M.; Two way ANOVA; P values *<0.05, **<0.005, ***<0.0005, ****<0.00005).

FIG. 1H shows the quantification of differentiation markers EOMES, KLRG1 and CD127. (CRE n=4, 1BKO n=2, TCKO n=4, TCKO/1BHet n=2, dKO n=4) (Bars represent t S.E.M.; Two way ANOVA; P values *<0.05, **<0.005, ***<0.0005, ****<0.00005).

FIG. 2A illustrates the conditional deletion of TCPTP and PTP1B in CD8 T cells. Shown is an immunoblot for TCPTP, PTP1B and Calnexin as loading control from protein homogenates of CD4 and CD8 T cells with different genotypes analyzed.

FIG. 2B illustrates the thymus size, expressed as weight, from 8-12-week-old male mice carrying the different genotypes analyzed.

FIG. 2C illustrates the hematoxylin and eosin staining of thymus sections from 10-week-old CRE controls or TCPTP/PTP1B (dKO) CD8 specific “knockouts”.

FIG. 2D shows spleen and inguinal lymph node size (by weight) and cellularity from the different genotypes analyzed.

FIG. 2E shows the total number of CD4 and CD8 T cells from spleen and inguinal lymph nodes shown in FIG. 1B.

FIG. 3A illustrates that mouse CD8 T cells deficient in TCPTP and/or PTP1B are more cytotoxic and reach terminal differentiation. OT-1 CD8 T cells with different Ptpn1 and/or Ptpn2 deletions were activated/expanded with αCD3/αCD28 antibodies and IL-2. Day 5 cells were tested for specific cytotoxicity against the OVA expressing E.G7 thymoma cells.

FIG. 3B shows statistical representation of several experiments from FIG. 3A.

FIG. 3C shows ELISA quantification of IFN-γ in supernatants from FIG. 3A.

FIG. 3D shows flow cytometry plots of CD8 T cell differentiation determined by CD62L and CD44 expression.

FIG. 3E shows bar representation of percentages of CD8 T cell differentiation determined by CD62L and CD44 expression.

FIG. 4 illustrates the in vitro cytotoxicity against specific target cells of CD8 T cells with genetic deficiency of TCPTP and/or PTP1B Statistical representation of day 5 CTLs from all the different genotypes tested for specific cytotoxicity against the OVA expressing E.G7 thymoma cells.

FIG. 5A Illustrates that pharmacological inhibition of TCPTP and PTP1B results in enhanced cytotoxicity and effector differentiation. CD8 T cells from OT-1 mice were activated/expanded by αCD3/αCD28 antibodies and IL-2 in the presence of different concentrations of TCPTP/PTP1B inhibitor (Inh1) for 5 days. Specific cytotoxicity against OVA expressing E.G7 thymoma cells was measure.

FIG. 5B shows the statistical representation of several experiments as per FIG. 5A (n=4).

FIG. 5C shows the ELISA quantification of IFN-γ in supernatants from FIG. 5A.

FIG. 5D shows flow cytometry plots of CD8 T cell differentiation determined by CD62L and CD44 expression.

FIG. 5E shows bar representation of percentages of CD8 T cell differentiation determined by CD62L and CD44 expression.

FIG. 5F shows the cytotoxicity of CD8 T cells from animals with different TCPTP and PTP1B genotypes, additionally treated with Ihn1.

FIG. 5G shows ELISA quantification of IFN-γ in supernatants from FIG. 5F. In each panel bars represent ±S.E.M.; Two-way ANOVA; P values *<0.05, **<0.005, ***<0.0005, ****<0.00005).

FIG. 6A illustrates that pharmacological inhibition of TCPTP and PTP1B in human T cells results in enhanced proinflammatory cytokine secretion and Tcm differentiation. Purified T cells from human donors were activated by αCD3/αCD28 and analyzed at day 7 for IFN-γ and TNF-α by flow cytometry. Shown are plots of cells gated in CD4 and CD8 populations distributed by IFN-γ and TNF-α expression.

FIG. 6B shows bar graphs showing statistical representation of 2 biological replicates for CD4 and CD8 populations distributed by IFN-γ expression.

FIG. 6C shows bar graphs showing statistical representation of 2 biological replicates for CD4 and CD8 populations distributed by TNF-α expression.

FIG. 6D shows bar graphs presenting the percentage of Effector Memory T cell (Tem) and Central memory T cell (Tcm) based on the selection of CD45 RO high and CD62L high ratio with or without inh1 in CD4 and CD8 T cells.

FIG. 7A illustrates the pharmacological inhibition of TCPTP and PTP1B in human T cells. Normalized percentual representation of experiments in FIG. 5 showing IFN-γ.

FIG. 7B illustrates the pharmacological inhibition of TCPTP and PTP1B in human T cells. Normalized percentual representation of experiments in FIG. 5 showing TNF-α.

FIG. 7C shows the percentual contribution of CD4 and CD8 T cells of in vitro human T cell cultures at day 7.

FIG. 8A illustrates the bulk mRNA analysis of CD8 T cells deficient in TCPTP and/or PTP1B or treated with Inh1. Heatmap of genes differentially expressed in the genotypes analyzed is shown. Organized as opposing expression between TCKO/1BHet CD8 T cells and CRE controls. Overexpressed genes showed as gradual red shift, under expressed genes as gradual blue shift.

FIG. 8B illustrates a volcano plot illustrating the significant differentially expressed genes between TCKO and TCKO/1B Het CD8 cells (FDR 5%).

FIG. 8C illustrates a dot plot corresponding to selected list of genes with relevancy to CD8 T cells. activation/differentiation. Overexpressed genes showed as gradual red shift, under expressed genes as gradual blue shift.

FIG. 9A illustrates bulk mRNA analysis of CD8 T cells deficient in TCPTP and/or PTP1B or treated with Inh1. Volcano plots representing the significant differentially expressed genes between day 5 CTLs from CRE controls and the different genotypes analyzed.

FIG. 9B illustrates the significant differentially expressed genes between day 5 CTLs form C57BL/6 mice, treated or not with 5 μM Inh1.

FIG. 9C illustrates the differential expression of the different members of the NR4A transcription factor family from samples in A) and B).

FIG. 9D illustrates the secretion of IL-10 from CTLs carrying different TCPTP and PTP1B mutations, treated or not with 5 μM Inh1. Determined by ELISA from supernatants form FIG. 1F.

FIG. 10A illustrates the global changes of STAT expression and phosphorylation in CD8T cells with reduced or absent TCPTP/PTP1B activity. Shown is an immunoblot of day 5 CTLs stimulated with rmIFN-β for the indicated times.

FIG. 10B illustrates the global changes of STAT expression and phosphorylation in CD8T cells with reduced or absent TCPTP/PTP1B activity. Shown is an immunoblot of day 5 CTLs stimulated with rmIFN-β for the indicated times.

FIG. 10C illustrates the global changes of STAT expression and phosphorylation in CD8T cells with reduced or absent TCPTP/PTP1B activity. Shown is a representation of densitometric analysis. (Representative of at least 3 experiments).

FIG. 11A illustrates the increased expression of transcription factors in CD8T cells with reduced or absent TCPTP/PTP1B activity. Shown is the specific cytotoxicity against OVA expressing E.G7 thymoma cells from 5-day TCKO/1BHet CTLs transfected with no target or BATF3 siRNAs.

FIG. 11B illustrates an immunoblot with specific BATF3 and calnexin antibodies to demonstrated specific deletion.

FIG. 11C illustrates an immunoblot analysis of cell homogenates from the different CTL genotypes analyzed, probed with antibodies for several transcription factors.

FIG. 12A illustrates that TCPTP/PTP1B pharmacological inhibitors have similar affinity for both enzymes and that intratumoral injection of Inh1 results in tumor regression. Shown is a timeline of tumor volume after subcutaneous injection of 3×10⁵ EG.7 OVA expressing thymoma cells in C57BL/6 mice. Mice were subjected to treatment by intratumoral injection of equivalent to 50 μM Inh1 every 2 days, or PBS as control. (Shown one of two experiments).

FIG. 12B illustrates the in vitro phosphatase activity of TCPTP and PTP1B catalytic domains in presence of increasing concentrations of Inh1 and Inh2. Table at right shows average IC₅₀ from 3 experiments.

FIG. 12C illustrates the specific cytotoxicity against OVA expressing E.G7 thymoma cells from 5-day C57BL/6 CTLs treated with Inh1 or Inh2 at the concentrations specified. (one of three experiments shown).

FIG. 13A illustrates that the TCPTP/PTP1B pharmacological inhibition synergize with anti-PD-1 antibodies to restrict B16F10 melanoma tumor growth in vivo. Shown is a timeline of tumor volume after subcutaneous injection of 1.5×10⁵B16F10 melanoma cells in C57BL/6 mice. Mice were subjected to treatment either with anti-PD-1 mAb (α-PD1) (n=3), TCPTP/PTP1B inhibitor (Inh2) (n=5), both (n=5) or none (n=5).

FIG. 13B illustrates the statistical comparison of all groups for experiment A) (Tukey's multiple comparison test with 95% confidence intervals).

FIG. 13C illustrates survival curve determined by humane endpoint intervention. (Data from one of two independent experiments).

FIG. 14A illustrates an immunoblotting analysis of Stat1 phosphorylation in PBMC of C57BL/6 mice after 2 hours of intraperitoneal injection of Inh2.

FIG. 14B illustrates an immunoblotting analysis of Stat3 phosphorylation in PBMC of C57BL/6 mice after 2 hours of intraperitoneal injection of Inh2.

### DETAILED DESCRIPTION

Compounds of Formula I and/or Formula II are inhibitors of both PTPN1 (PTP1B) and PTPN2 (TCPTP). T lymphocytes deficient in TCPTP, or deficient in TCPTP and additionally hemi-deficiency of PTP1B display enhanced pro-cytotoxic abilities. Small molecule inhibitors targeting specifically both the PTP1B and TCPTP enzymes showed a remarkable enhancement of treated CD8 T cells in mouse and human cells, providing therapeutic use in solid tumors.

According to an embodiment, the present invention relates to a method for the treatment of cancer by administering a therapeutically effective amount of a compound of structural Formula I, of structural Formula II, or pharmaceutically acceptable salts thereof, and stereoisomers thereof, or combinations thereof, in combination with an immune checkpoint inhibitor, to a subject in need thereof.

According to another embodiment, the present invention relates to a method for the treatment of PTP1B and TCPTP dependent diseases, such as for example cancer, by administering a therapeutically effective amount of a compound of structural Formula I, of structural Formula II, or pharmaceutically acceptable salts thereof, and stereoisomers thereof, or combinations thereof, in combination with an immune checkpoint inhibitor, to a subject in need thereof.

Types of cancer that may be treated by compounds of the present invention include, but are not limited to, prostate cancer, breast cancer, brain cancer, glioma, lung cancer, salivary cancer, stomach cancer, thymic epithelial cancer, thyroid cancer, ovarian cancer, multiple myeloma, leukemia, melanoma, lymphoma, gastric cancer, kidney cancer, pancreatic cancer, bladder cancer, colon cancer and liver cancer.

### Abbreviations

Abbreviations and terms that are commonly used in the fields of organic chemistry, medicinal chemistry, pharmacology, and medicine and are well known to practitioners in these fields are used herein. Representative abbreviations and definitions are provided below:

Ac is acetyl [CH₃C(O)—], Ac₂O is acetic anhydride; ACN is acetontrile; APC is antigen-presenting cell; Alk is alkyl; Ar is aryl; 9-BBN is 9-borabicyclo[3.3.1]nonane; Bn is benzyl; BOC is tert Butyloxycarbonyl; br is broad; CH₂Cl₂ is dichloromethane; d is doublet; DBU is 1,8-diazabicyclo[5.4.0]undec-7-ene; DC is dendritic cell; DEAD is diethyl azodicarboxylate; DIAD is diisopropylazodicarboxylate; DIBAL is diisobutylaluminum hydride; DIPEA is N,N-diisopropylethylamine; DMF is N,N-dimethylformamide; DMSO is dimethyl sulfoxide; EDAC (or EDC) is 1-ethyl-3-[3-(dimethylamino)propyl]-carbodiimide HCl; ESI is electrospray ionization; Et₃N is triethylamine; Et is ethyl; EtOAc is ethyl acetate; EtOH is ethanol; 3-F-Ph is 3-fluorophenyl; h is hours; HATU is O-(7-azabenzotriazol-1-yl)-N,N,N′,N′-tetramethyluronium hexafluorophosphate; HOAc is acetic acid; HCl is hydrochloric acid; HOBt is 1-hydroxybenzotriazole; HPLC is high performance liquid chromatography; Hunig's base is diisopropylethylamine; LiOH is lithium hydroxide; LCMS is HPLC with mass Spectral detection; LG is leaving group; m is multiplet; M is molar; mmol is millimole; Me is methyl; MeCN is acetonitrile; MeOH is methanol; MeTHF is 2-methyltetrahydrofuran; MgSO₄ is magnesium sulfate; min is minutes; MS is mass spectroscopy; MsCl is methanesulfonyl chloride; MTBE is methyl tert-butyl ether; N is normal; NaHMDS is sodium hexamethyldisiliazide; NaOAc is sodium acetate; NaOH is sodium hydroxide; NaOtBu is sodium tert-butoxide; Na₂SO₄ is sodium sulfate; NMO is N-methylmorpholine N oxide; NMP is N Methyl pyrrolidinone; NMR is nuclear magnetic resonance spectroscopy; Pd(dba)₂ is tris(dibenzylideneacetone)dipalladium; PdCl₂(Ph₃P)₂ is dichlorobis-(triphenylphosphene) palladium; PG Denotes an unspecified protecting group; Ph is phenyl; PhMe is toluene; PPh₃ is triphenylphosphine; PMB is para-methoxybenzyl; RT is room temperature; s is singlet; t is triplet; TBAF is tetrabutyl ammonium fluoride; TBS is tert-butyldimethylsilyl; tBu is tert-butyl; Tf is triflate; TFA is trifluoroacetic acid; TFAA is trifluoroacetic anhydride; THF is tetrahydrofuran; TLC is thin layer chromatography; TMEDA is N,N,N′N′-tetramethylethylenediamine; TMS is trimethylsilyl; TPAP is tetrapropylammonium perruthenate.

### Definitions

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C3-10, the term alkyl also includes cycloalkyl groups, and combinations of linear or branched alkyl chains combined with cycloalkyl structures. When no number of carbon atoms is specified, C1-6 is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C1-6 alkoxy), or any number within this range [i.e., methoxy (MeO—), ethoxy, isopropoxy, etc.].

The term “alkylthio” refers to straight or branched chain alkylsulfides of the number of carbon atoms specified (e.g., C1-6 alkylthio), or any number within this range [i.e., methylthio (MeS—), ethylthio, isopropylthio, etc.].

The term “alkylamino” refers to straight or branched alkylamines of the number of carbon atoms specified (e.g., C1-6 alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C1-6 alkylsulfonyl), or any number within this range [i.e., methylsulfonyl (MeSO₂⁻), ethylsulfonyl, isopropylsulfonyl, etc.].

The term “alkylsulfinyl” refers to straight or branched chain alkylsulfoxides of the number of carbon atoms specified (e.g., C1-6 alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO⁻), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

The term «composition» as used herein is intended to encompass a product comprising the specified ingredients in the specified amounts, as well as any product which results, directly or indirectly, from combination of the specified ingredients in the specified amounts. Such term in relation to pharmaceutical composition is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

The terms «T cell(s)», «T lymphocyte(s)», «T cell product(s)» as used herein are intended to encompass isolated tumor-infiltrating lymphocyte (TIL), T cell receptor (TCR) engineered cell, and/or chimeric antigen receptor (CAR) engineered cell isolated by the method of the present invention. It also includes different memory T cell populations such as Stem central memory T_{SCM} cells, Central memory T_{CM} cells and Effector memory T_{EM} cells, that are beneficial to mount and maintain surveillance and Immune response.

The terms “PTPN1” as used herein is intended to mean the tyrosine-protein phosphatase non-receptor type 1, also known as protein-tyrosine phosphatase 1B (PTP1B), and is an enzyme that is the founding member of the protein tyrosine phosphatase (PTP) family. In humans it is encoded by the PTPN1 gene. PTP1B is a negative regulator of the insulin signaling pathway and is considered a promising potential therapeutic target, in particular for treatment of type 2 diabetes. It has also been implicated in the development of breast cancer and has been explored as a potential therapeutic target in that avenue as well.

The terms “PTPN2” as used herein is intended to mean the tyrosine-protein phosphatase non-receptor type 2, also known as T-cell protein-tyrosine phosphatase (TCPTP, TC-PTP), and in humans is encoded by the PTPN2 gene.

### Compounds of Structural Formula I and Formula II

The present invention uses compounds of structural Formula I, or of structural Formula II, or pharmaceutically acceptable salts thereof, and stereoisomers thereof, or combinations thereof. The compounds structural Formula I are the following:

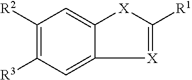

wherein:
X is selected from CH and N;
R¹ is selected from the group consisting of (a) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) —(C═O)R⁴; (c) —CN; (d) —(C═O)OR⁴; (e) —(C═O)NHR⁴; (f) —(C═O)NR⁵R⁶; and (g) aryl or heteroaryl wherein the aryl and heteroaryl group itself may be optionally substituted with 1-3 substituents independently selected from (i) halogen, (ii) —(C═O)OC₁₋₃alkyl optionally substituted with 1-3 halogens, (iii) —COOH (iv) C₁₋₃ alkyl optionally substituted with 1-3 halogens, (v) —OC₁₋₃ alkyl optionally substituted with 1-3 halogens, (vi) —SOₓMe, (vii) —CN, and (viii) —SO₂NH₂;
R² and R³ are independently selected from the group consisting of (a) halogen; (b) difluoromethylphosphonic acid;
R⁴ is selected from the group consisting of (a) H; (b) C₁₋₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (d) aryl or heteroaryl wherein the aryl or heteroaryl group itself may be optionally substituted by 1-3 halogens, C₁₋₃ alkyl or C₁₋₃ haloalkyl;
R⁵ and R⁶ are independently selected from the group consisting of (a) C₃ alkyl optionally substituted with 1-5 halogens and optionally with one group selected from —OH, —OC₁₋₃ alkyl optionally substituted with 1-3 halogens, —SOₓC₁₋₃ alkyl, and —CN; (b) aryl or heteroaryl wherein the aryl or heteroaryl group itself may be optionally substituted by 1-3 halogens, C₁₋₃ alkyl or C₁₋₃ haloalkyl;
R⁵ and R⁶, together with the nitrogen atom to which they are attached may be joined to form a 5- to 7-membered ring, which may be substituted with 1-3 groups independently selected from (i) halogen, (ii) —(C═O)OC₁₋₃ alkyl, (iii) —(C═O)OH (iv) C₁₋₃ alkyl optionally substituted with 1-3 halogens, (v) —OC₁₋₃ alkyl optionally substituted with 1-3 halogens, (vi) —OH, (vii) C₁₋₃ hydroxyalkyl, (viii) aryl or heteroaryl wherein the aryl or heteroaryl group itself may be optionally substituted by 1-3 halogens, C₁ 3 alkyl or CO₁₃ haloalkyl; and
x is an integer from 0 to 2.

The compounds of structural formula I include compounds of structural Formula Ia, or a pharmaceutically acceptable salts thereof, and stereoisomers thereof:

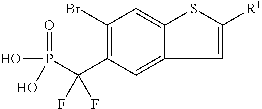

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of CO₁₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compounds of structural formula I include compounds of structural Formula Ib, or a pharmaceutically acceptable salts thereof, and stereoisomers thereof:

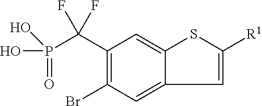

wherein:
R¹ is selected from the group consisting of (a) C₁₋₃ alkyl optionally substituted with 1-5 halogens and optionally with one group selected from —OH, —OC₁₋₃ alkyl optionally substituted with 1-3 halogens, and —CN; (b) —(C═O)R⁴; (c) —CN; (d) —(C═O)OR⁴; (e) —(C═O)NHR⁴; and (f) —(C═O)NR⁵R⁶;
R⁴ is selected from the group consisting of (a) H; and (b) C₁₋₃ alkyl optionally substituted with 1-5 halogens;
R⁵ and R⁶ are independently selected from the group consisting of C₁₋₃ alkyl optionally substituted with 1-5 halogens and optionally with one group selected from —OH, and —OC₁₋₃ alkyl optionally substituted with 1-3 halogens; and
R⁵ and R⁶, together with the nitrogen atom to which they are attached may be joined to form a 5- to 7-membered ring, which may be substituted with a 1-3 groups independently selected from (i) halogen, (ii) C₁₋₃ alkyl optionally substituted with 1-3 halogens, (iii) —OC₁₋₃ alkyl optionally substituted with 1-3 halogens, (iv) —OH, and (vii) C₁₋₃ hydroxyalkyl.

The compounds of structural Formula I may be compounds selected from the following compounds:

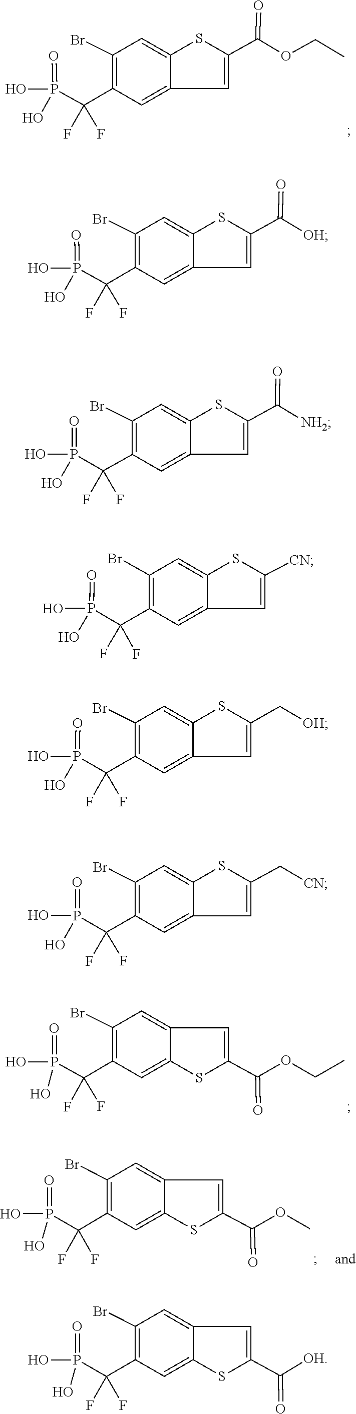

The compounds structural Formula II are the following:

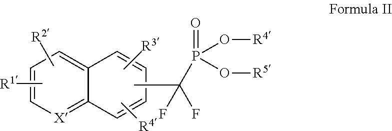

wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (I) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group, R^{4′} and R^{5′} are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
(i) —(C═O)OR⁷,
(ii) —(C═O)NHR⁷,
(iii) —(C═O)N(R⁷)₂,
(iv) —(C═O)NH₂,
(v) —OR⁷,
(vi) —O(C═O)R⁷,
(vii) —O(C═O)OR⁷,
(viii) —O(C═O)NHR⁷,
(ix) —O(C═O)N(R⁷)₂,
(x) —O(C═O)NH₂,
(xi) —SO₂NH₂,
(xii) —SOₓCH₃,
(viii) —S(C═O)R⁷ and
(ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R^{4′} and R⁵ together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl; and
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R⁷ is selected from the group consisting of C₁₋₆ alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃ alkyl;
R^{a} and R^{b} are each independently hydrogen or C₁₋₄ alkyl optionally substituted with hydroxy or 1-5 fluorines; and
x is 0, 1, or 2;

The compound of formula II may be of structural Formula IIa, or a pharmaceutically acceptable salts thereof, and stereoisomers thereof:

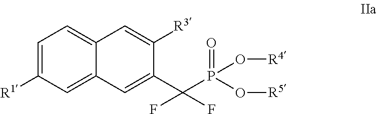

wherein
R^{1′} is selected from the group consisting of (a) C₁₋₃ alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —HC═NOH, (e) —(CH₃)C═NOH, (f) —HC═NOC₁₋₃alkyl optionally substituted with 1-3 halogens, (g) —(CH₃)C═NOC₁₋₃alkyl optionally substituted with 1-3 halogens, (h) —C(═O)OC₁₋₃alkyl optionally substituted with 1-3 halogens, (i) —C(═O)NHR^{6′}, (j) —CH═CH-Phenyl wherein —CH═CH— is optionally substituted with 1-2 substituents independently selected from halogen and C₁₋₂alkyl optionally substituted with 1-3 F, (k) —CH₂CH₂-Phenyl wherein —CH₂CH₂— is optionally substituted with 1-4 substituents independently selected from halogen and C₁₋₂alkyl optionally substituted with 1-3 F, (I) Phenyl, (m) -HET-Phenyl, wherein HET is a 5- or 6-membered heteroaromatic ring containing 1-3 heteroatoms selected from O, N and S, (n) —C≡C-Phenyl, (o) —CH₂-Phenyl, and (p) —CN, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{3′} is halogen;
R⁴ and R⁵ are each independently selected from the group consisting of:
(a) hydrogen:
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
   (i) —(C═O)OR⁷,
   (ii) —(C═O)NHR⁷,
   (iii) —(C═O)N(R⁷)₂,
   (iv) —(C═O)NH₂,
   (v) —OR⁷,
   (vi) —O(C═O)R⁷,
   (vii) —O(C═O)OR⁷,
   (viii) —O(C═O)NHR⁷,
   (ix) —O(C═O)N(R⁷)₂,
   (x) —O(C═O)NH₂,
   (xi) —SO₂NH₂,
   (xii) —SOₓCH₃,
   (viii) —S(C═O)R⁷, and
   (xiii) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
   or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
   R^{6′} is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃alkyl optionally substituted with 1-3 halogens, (iii) —COOH, (iv) C₁₋₃ alkyl optionally substituted with 1-3 halogens, and (v) —OC₁₋₃ alkyl optionally substituted with 1-3 halogens;
   R⁷ is selected from the group consisting of C₁₋₆ alkyl optionally substituted with 1-3 substituents independently selected from (i) halogen, (ii) —OC₁₋₃ alkyl, (iii) aryl, and (iv) heteroaryl, wherein the aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃alkyl;
R^{a} and R^{b} are each independently hydrogen or C₁₋₄ alkyl optionally substituted with hydroxy or 1-5 fluorines; and
   x is 0, 1, or 2.

The compound of structural Formula II may be selected from the following compounds:

| Ex. | Structure |
|---|---|
| 2 | 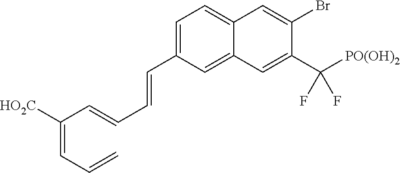 |
| 3 | 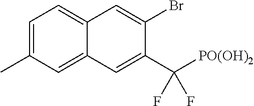 |
| 4 | 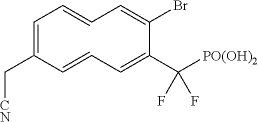 |
| 5 | 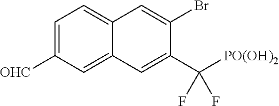 |
| 6 | 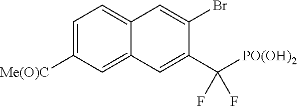 |
| 8a | 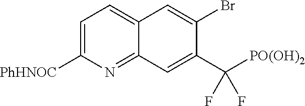 |
| 8b | 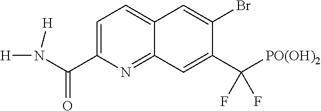 |
| 8c | 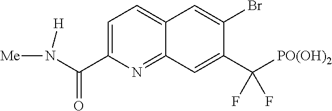 |
| 8d | 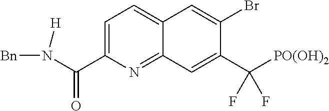 |
| 8e | 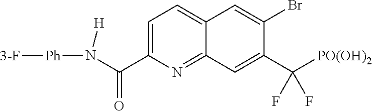 |
| 9a | 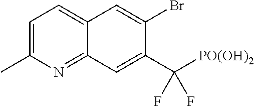 |
| 9c | 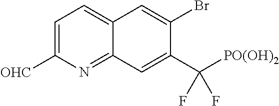 |
| 9d | 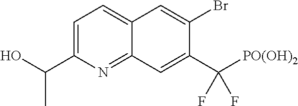 |
| 9e | 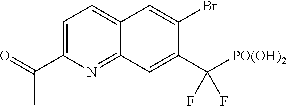 |
| 9f | 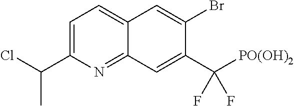 |
| 9g | 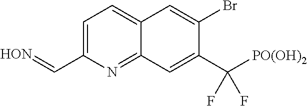 |
| 9h | 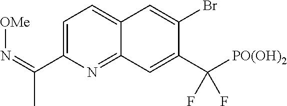 |
| L598 | 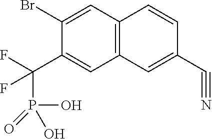 [SMILES: N#Cc1ccc2cc(Br)c(C(F)(F)P(=O)(O)O)cc2c1] |
| 10 | 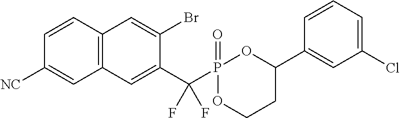 [SMILES: N#Cc1ccc2cc(Br)c(C(F)(F)P3(=O)OCCC(c4cccc(Cl)c4)O3)cc2c1] |
| 11 | 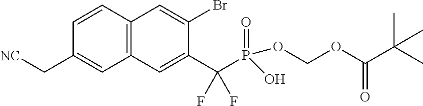 [SMILES: CC(C)(C)C(=O)OCOP(=O)(O)C(F)(F)c1cc2cc(CC#N)ccc2cc1Br] |
| 12 | 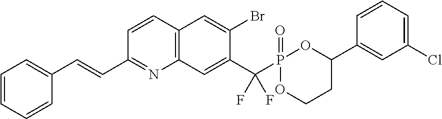 [SMILES: O=P1(C(F)(F)c2cc3nc(/C=C/c4ccccc4)ccc3cc2Br)OCCC(c2cccc(Cl)c2)O1] |
| 13 | 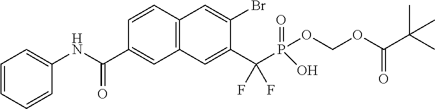 [SMILES: CC(C)(C)C(=O)OCOP(=O)(O)C(F)(F)c1cc2cc(C(=O)Nc3ccccc3)ccc2cc1Br] |
| 14 | 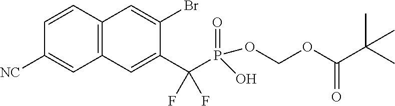 [SMILES: CC(C)(C)C(=O)OCOP(=O)(O)C(F)(F)c1cc2cc(C#N)ccc2cc1Br] |
| 15 | 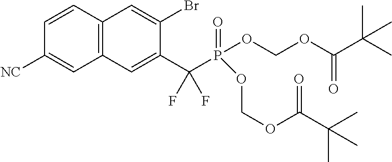 [SMILES: CC(C)(C)C(=O)OCOP(=O)(OCOC(=O)C(C)(C)C)C(F)(F)c1cc2cc(C#N)ccc2cc1Br] |

or a pharmaceutically acceptable salt thereof.

Compounds of structural Formula I, structural Formula Ia and/or structural Formula Ib and/or structural Formula II and/or structural Formula IIa may contain one or more asymmetric centers and can thus occur as racemates and racemic mixtures, single enantiomers, diastereomeric mixtures and individual diastereomers. The present invention is meant to comprehend all such isomeric forms of the compounds of structural Formula I, structural Formula Ia and/or structural Formula Ib.

Compounds of structural Formula I, structural Formula Ia, structural Formula Ib and/or structural Formula II and/or structural Formula IIa may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural Formula I, structural Formula Ia, structural Formula Ib and/or structural Formula II and/or structural Formula IIa may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, Formula Ia, Formula Ib, Formula II and/or Formula IIa, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I, Formula Ia, Formula Ib and/or structural Formula II and/or structural Formula IIa. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I, Formula Ia, Formula Ib, Formula II and/or Formula IIa can be prepared without undue experimentation by conventional techniques well known to those skilled in the art or by processes analogous to those described in the Schemes and Examples herein using appropriate isotopically-enriched reagents and/or intermediates.

The term “subject” as used herein, is a human patient or other animal such as another mammal with functional mast cells, basophils, neutrophils, eosinophils, monocytes, macrophages, dendritic cells, and Langerhans cells.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

### Salts and Formulations

It will be understood that, as used herein, references to the compounds of structural Formula I, Formula Ia, Formula Ib, Formula II and/or Formula IIa are meant to also include the pharmaceutically acceptable salts, and also salts that are not pharmaceutically acceptable when they are used as precursors to the free compounds or their pharmaceutically acceptable salts or in other synthetic manipulations. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural Formula I, Formula Ia, Formula Ib, Formula II and/or Formula IIa are included in the present invention as well.

The pharmaceutical compositions may be in the form of a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

### Immune Checkpoint Inhibitors

The role of immune checkpoints is to prevent an immune response from being so strong that it destroys healthy cells in the body. Immune checkpoints engage when proteins on the surface of T cells recognize and bind to partner proteins on other cells, such as some tumor cells. These proteins are called immune checkpoint proteins. When the checkpoint and partner proteins bind together, they send an “off” signal to the T cells. This can prevent the immune system from destroying the cancer.

Immunotherapy drugs called immune checkpoint inhibitors work by blocking checkpoint proteins from binding with their partner proteins. This prevents the “off” signal from being sent, allowing the T cells to kill cancer cells. One such drug acts against a checkpoint protein called CTLA-4. Other immune checkpoint inhibitors act against a checkpoint protein called Programmed cell death protein 1, also known as PD-1 or its partner protein Programmed death-ligand 1 (PD-L1). Some tumors turn down the T cell response by producing lots of PD-L1.

The most recognized checkpoint inhibitors are anti-PD1 and anti-CTLA4, yet in the interaction of dendritic cells with tumors cells, effector T-cells, and other immune cells, a number of protein interactions favoring or inhibiting the recognition and killing of tumor cells has been identified. For example, a dozen of those interactions have been reported to affect DC and tumors cells (K. Palucka and J. Banchereau, Nature Reviews Cancer 12:265-277). Hence the technology described herein may be conjugated to many of those additional immunotherapy technologies currently in development.

According to embodiment, the immune checkpoint inhibitor may therefore be an anti-PD-1 antibody, an anti-PD-L1 antibody, an anti-CTLA-4 antibody, or molecular inhibitors of these receptors.

The anti-PD-1 antibody may be selected from the group consisting of nivolumab antibody, pembrolizumab antibody, pidilizumab antibody, cemiplimab antibody, dostarlimab antibody, JTX-4014 antibody, Spartalizumab antibody, Camrelizumab antibody, Sintilimab antibody, Tislelizumab antibody, Toripalimab antibody, INCMGA00012 antibody, AMP-224 antibody, AMP-514 antibody, RMP1-14 antibody or combinations thereof.

The anti-PDL-1 antibody may be selected from the group consisting of B7-H1 antibody, BMS-936559 antibody, MPDL3280A (atezolizumab) antibody, MEDI-4736 antibody, MSB0010718C antibody, 10F-9G2 antibody, avelumab antibody, durvalumab antibody, or combinations thereof.

The anti-CTLA-4 antibody may be selected from the group consisting of ipilimumab or tremelimumab or combinations thereof.

### Utilities

The compounds specifically exemplified herein exhibit good efficacy in inhibiting the PTPN1/PTP1B and PTPN2/TCPTP enzymes, as shown by their in vitro assays. The compounds generally have an IC₅₀ value of less than 10 μM in the enzyme assay described in the Assays section, and preferably have an IC₅₀ value of less than 1 μM.

One aspect of the invention provides a method for the treatment and control of cancer, which comprises administering to a patient in need of such treatment a therapeutically effective amount of a compound of Formula I, Formula Ia, Formula Ib, Formula II and/or compounds of Formula IIa, in combination with an immune checkpoint inhibitor. Combined together, the compound of Formula I, Formula Ia, Formula Ib, Formula II and/or compounds of Formula IIa and the immune checkpoint inhibitor provide an unexpectedly enhanced treatment that may not be obtained from the treatment with either component alone.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, can be treated. However, the method can also be practiced in other species, such as avian species (e.g., chickens).

### Kits

Compounds of Formula I, Formula Ia, Formula Ib, Formula II and Formula IIa when being used for treatment purposes, may be packaged for use as a crystalline solid, an amorphous solid or a lyophilized powder. Suitable quantities range from about 0.1 mg to 1 g. Ideally, the compound is packaged in a container to which a suitable solvent can be added to achieve the desired concentration of solution. Alternatively, the compound may be packaged as an aqueous solution at a fixed concentration, or as a solution in a water-soluble organic solvent at a fixed concentration. Suitable organic solvents may include DMSO, methanol, ethanol or acetonitrile, or mixtures of these solvents with water. Suitable concentrations are about 0.1 mM to about 25 mM.

The present invention includes kits encompassing the compounds of Formula I, Formula Ia, Formula Ib, formula II and/or Formula IIa, and instructions on how to use said compounds. According to an embodiment, the kit may also include appropriate immune checkpoint inhibitor. The kit will allow a patient to be conveniently treated. This treatment can be optimized to work best with current clinical therapeutic standards.

The compound of Formula I, Formula Ia, Formula Ib, Formula II and/or Formula IIa may be administered to a patient in need of immunotherapy in one or more injections. The frequency of injection and the intervals between injections of the compound and/or immune checkpoint inhibitor will be adjusted to maximize the therapeutic response. For example, injections may occur once, twice, or more times daily, once, twice, or more times weekly, biweekly, monthly or bimonthly or at any other intervals deemed most suitable to the therapeutic benefit of the patient. The compound of Formula I, Formula Ia, Formula Ib, Formula II and/or Formula IIa may be administered prior to administration of the immune checkpoint inhibitor.

The administration of the compound of Formula I, Formula Ia, Formula Ib, Formula II and/or Formula IIa may be an intratumoral administration. The administration of the compound of structural Formula I, Formula Ia, Formula Ib, Formula II and/or Formula IIa may be an oral administration. The administration of the immune checkpoint inhibitor may be a systemic administration, and intratumoral administration, or a combination thereof.

The administration of said compound of structural Formula I, Formula Ia, Formula Ib, Formula II and/or Formula IIa may be an intratumoral administration and the administration of the immune checkpoint inhibitor may be a systemic administration, an intratumoral administration, or a combination thereof, or the administration of the compound of structural Formula I may be an intratumoral administration and administration of the immune checkpoint inhibitor may be a systemic administration, an intratumoral administration, or a combination thereof, or administration of the compound of structural Formula I may be an oral administration and administration of the immune checkpoint inhibitor may be a systemic administration, an intratumoral administration, or a combination thereof.

### Combination Therapy

A patient in need of immunotherapy may be treated with the compound of Formula I, Formula Ia, Formula Ib, Formula II and/or Formula IIa and the immune checkpoint inhibitor, contemporaneously with other treatments known to the medical practitioner. The use of such multiple treatments may be particularly advantageous to the patient. Such treatments may include, but are not limited to, surgical resection, radiation, chemotherapy, targeted therapy and other types of immunotherapy. Chemotherapy agents that may be used include:
a) cytotoxic agents such as taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof;
b) antimetabolites such as methotrexate, 6-mercaptopurine, 6-thioguanine, gemcitabine, cytarabine, 5-fluorouracil decarbazine;
c) alkylating agents such as mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin;
d) anthracyclines such as daunorubicin and doxorubicin;
e) antibiotics such as dactinomycin, bleomycin, mithramycin, and anthramycin (AMC); f) anti-mitotic agents such as vincristine and vinblastine;
g) targeted therapies that may be used include, but they are not limited to: hormone therapies (such as degarelix, a luteinizing hormone-releasing hormone (LHRH) antagonist that reduces testosterone levels in prostate cancer), signal transduction inhibitors (such as imatinib, trastuzumab, PARPi, and CDKi), as well as gene expression modulators (for example the HDAC inhibitors panobinostat and belinostat), apoptosis inducers (such as recombinant human TNF-related apoptosis-inducing ligand (TRAIL)) and angiogenesis inhibitors (such as sorafenib, sunitinib, pazopanib and everolimus);
h) Immunotherapy agents that may be used include: monoclonal antibodies treatment (anti-LAG3, anti-KIR), and dendritic cell (DC) vaccines.

### Monitoring

According to another embodiment, the methods or uses of the present invention may further comprise monitoring the activity of the compound of structural Formula I, structural Formula II or both structural Formula I and structural Formula II during their respective uses. For example, targeted cells, such as PBMCs can be collected from blood drawn from a patient treated with the compounds of the present invention, and the phosphorylation states of downstream targets of TCPTP (PTPN2) and PTP1B (PTPN1) may be monitored. As per examples below, the monitoring may take place by immunoblotting, but could also take place through other means of measuring the phosphorylation states of these downstream targets. For example, monitoring could be performed by measuring the fluorescence from a fluorescently labelled antibody specific to any one of the PTPN1/PTPN2 downstream targets, by Fluorescent activated Cell Sorting, mass spectrometry and others.

The present invention will be more readily understood by referring to the following examples which are given to illustrate the invention rather than to limit its scope.

### Example 1

### Conditional TCPTP and/or PTP1B Inactivation in Endogenous CD8+ T Cells of Mice

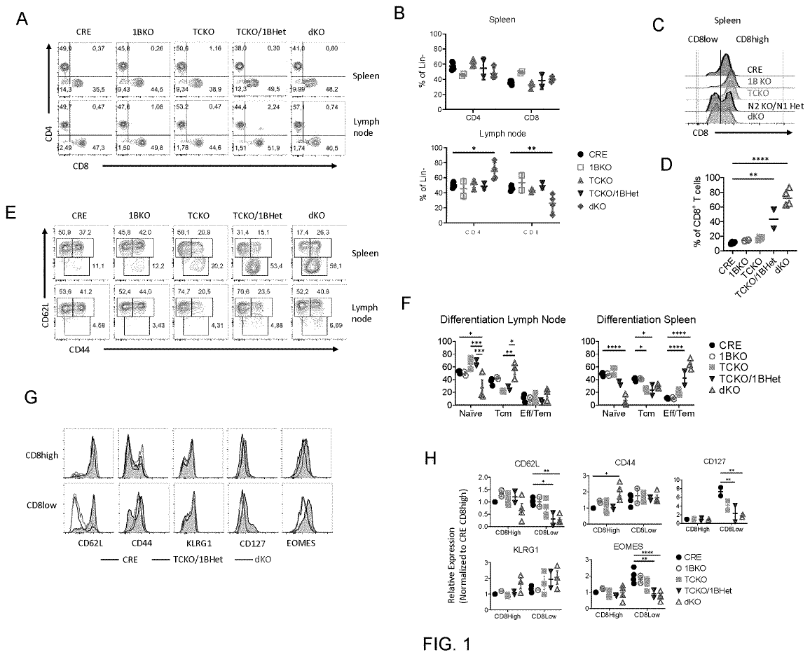

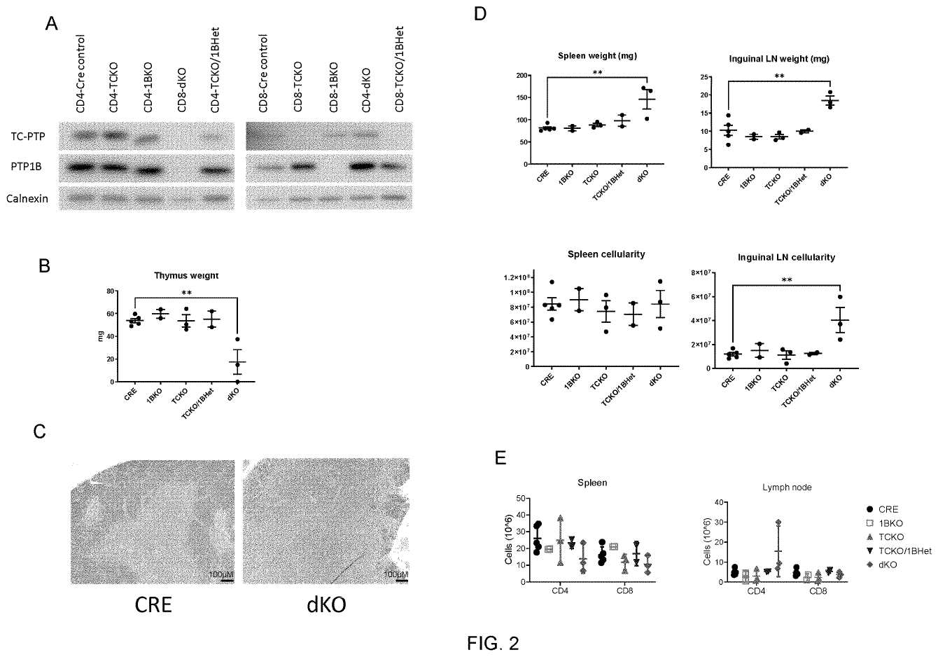

To address the combined contribution of TCPTP and PTP1B to the regulation of activating signals in CD8+ T cells, mouse lines where allelic inactivation of either one or both of these genes was specifically induced in mature CD8+ T cells. To achieve this, heterozygous mice carrying “floxed” alleles of both genes, TCPTP [30] and PTP1B [31], were bred with a mouse line where the CRE recombinase expression is driven by the CD8 enhancer 8I (E8i-CRE) [32]. Mice were bred to produce littermates with either WT (wt/wt), heterozygous (wt/fl) or homozygous (fl/fl) versions of either single TCPTP or PTP1B, or both genes. Throughout the experiments, heterozygous E8i-CRE (wt/cre+) expression was maintained to achieve deletion. To avoid accounting potential artifacts arising from off-target effects of the CRE recombinase [33, 34], mice heterozygous for E8i-CRE (wt/cre+) carrying wt alleles for TCPTP and PTP1B were used as controls, here after named CRE. Mice were born at mendelian rates, and no apparent differences were detected between the various genotypes until 6-8 weeks of age. CD8 T cells from all genotypes reached maturity and distributed in peripheral secondary immune organs (FIG. 1). To confirm that conditional deletion of both genes was achieved, protein expression of TC-PTP and PTP1B was tested by immunoblotting in both peripheral CD4 and CD8 T cells (FIG. 2). As expected, protein expression corresponded to each genotype and was not observed in CD4 T cells. Nevertheless, around 10 weeks of age mice lacking expression of both genes in CD8+ T cells (ptpn1 fl/fl; ptpn2 fl/fl; wt/cre+) or dKO hereafter, showed a significant reduction in thymus size with complete involution observed around 12 weeks of age (FIG. 2B). Microscopically the cortical and medullary architecture was loss in thymi of these mice (FIG. 2C) when compared to CRE controls. In the same mice, mild splenomegaly and spontaneous lymphadenopathies were observed. In contrast, other genotypes tested did not show differences in organ size when compared to CRE controls (FIG. 2D).

### Example 2

### Analysis of Circulating T Cells

Next, circulating CD8 populations from the different genotypes were analyzed by flow cytometric analysis. Although CD8 T cells were present in spleen and lymph nodes of all the genotypes analyzed, mice with dKO CD8 T cells showed an increase in the CD4/CD8 T cell ratio in lymph nodes. However, the absolute number of T cells was not significantly affected (FIGS. 1A, 1B and 2E). This finding, together with the size increase of lymph nodes suggested a change in the migratory cues of lymph nodes. No other significant differences in the distribution of T cells in lymph nodes or spleen was noted. Notwithstanding, subpopulation of CD8 T cells expressing low levels of the CD8 coreceptor were detected in the spleen of mice producing cells with the most extreme genotypes, dKO and TCKO/1BHet (ptpn1 wt/fl; Ptpn2 fl/fl; wt/cre+) T cells (FIGS. 1A and 1D). The accumulation of these CD8low expressing cells was more pronounced in the dKO animals, almost double the amount seen in the TCKO/1 BHet animals, comprising around 70% and 40% of all splenic CD8 T cells respectively (FIGS. 1C and 1D). Cells displaying this phenotype were also observed in CRE controls, and both single KO, however they were present at much lesser percentages (ranging from an average 13% to 17%) and differences were not statistically significant (FIGS. 1C and 1D). To further characterize peripheral CD8 T cells in these animals, their differentiation state was assessed by co-staining for the trafficking molecule CD62L and the memory marker CD44 (FIG. 1E). Different CD8 T cell subpopulations of the spleen and peripheral lymph nodes were sorted based on L-selectin (CD62L) and CD44 surface molecule expression defining naïve, effector/effector memory and central memory subpopulations (FIGS. 1E and 1F). While the differentiation state of CD8 T cells in lymph nodes seemed unaffected within the various genotypes, spleens showed a marked increase of effector/effector memory like CD8 T cells in the TCKO/1 BHet mice (ptpn1 wt/fl; Ptpn2 fl/fl; wt/cre+) and dKO mice. This subset of CD8+ T cells, characterized by reduced expression of CD62L and intermediate expression of CD44, correlated with the presence CD8low cells (FIG. 1D). To determine if this correlation was indeed obtained from a distinct differentiation state of the CD8low subpopulation the expression of known CD8 differentiation markers in these cells was compared with the CD8high subpopulation. Besides CD44 and CD62L, the expression of the transcription factor EOMES, the IL-7 receptor CD127 and the terminal differentiation marker KLRG1 were evaluated (FIGS. 1G and 1H). In general, it was found that CD8low cells from the TCKO/1 BHet and dKO mice, besides a strong effector phenotype given by low CD62L and increased CD44 expression, showed a terminal effector phenotype characterized by loss of CD127, a decrease in EOMES expression and a slight increase in KLRG1 expression. These phenotypical characteristics define this subpopulation as short live effector CD8 cells [35]. Hence, in these 2 genotypes CD8+ T cells tended to spontaneously differentiate into terminal effector cells with preferential migration to the spleen.

### Example 3

### Analysis of Functional Capabilities of CD8 T Cells

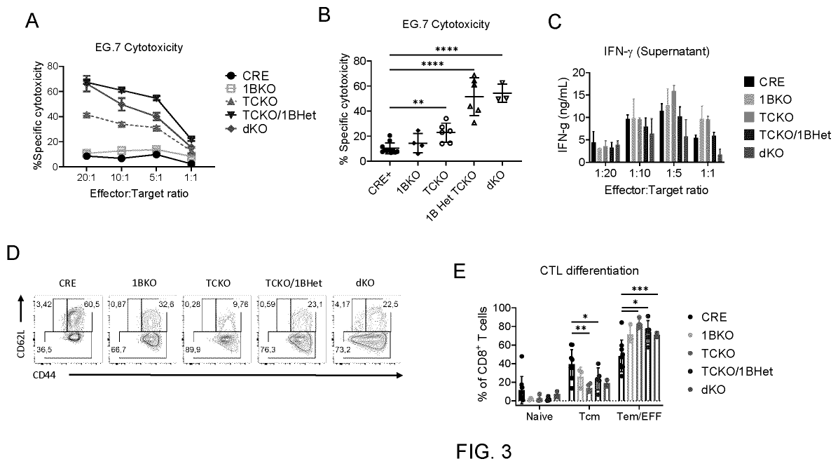

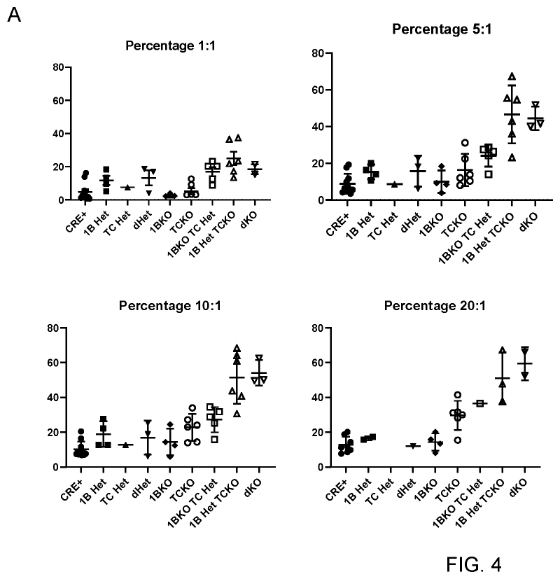

To understand the functional capabilities of the CD8 T cells with combined deficiency of TCPTP and PTP1B, the conditional mutant mice of Example 1 were bred with OT-1 transgenic mice. OT-1 mice express a T cell receptor (TCR) with affinity for an epitope of chicken ovalbumin (OVA 257-64) recognized in context of the MHC Class I. OVA specific activated cytotoxic lymphocytes (CTL) were generated in vitro from purified splenic CD8+ T cells of mice with different genotypes. Day 5 CTL cells were then challenged with the OVA expressing thymoma cell line E.G7 in 5-hour in vitro cytotoxicity assays (FIGS. 3A, 3B and 4A). Regarding the single mutant cells, an increase of the cytolytic activity of CTL deficient for TCPTP was observed, but not in the cells from mice lacking PTP1B, when compared to similarly treated CRE controls. The enhanced effector activity seen in the TCPTP deficient cells is in line with previous observations [20, 21]. Remarkably, a significant increase in the cytolytic activity of CTL from TCKO/1 BHet and dKO genotypes over those with sole TCPTP deficiency was consistently observed. This increase was evident at different effector to target ratios, being almost 2-fold at 5:1 and 10:1 ratios when compared to the TCKO (FIGS. 3A and 3B). Effector CD8 T cells also contribute to the immune response by the secretion of cytokines as IFN-γ, consequently the presence of this cytokine in the supernatant of target cell stimulated CTLs by ELISA was measured. An increase of the secretion of IFN-γ was detected in TCPTP and PTP1B single deficient cells at low effector to target ratios which was more stepped in the PTPN2 KO cells (FIG. 3C). However, a significant reduction of IFN-γ concentration was observed in the supernatants from dKO CTLs, more evident in low effector to target ratios while no significant changes were seen in the TCKO/1BHet, which were conversely the most efficient at tumor lysis (FIG. 3C). As CD8 differentiation dictates the functional state of these cells, analysis by flow cytometry of the expression of the CD62L and CD44 surface markers on this invitro stimulated CTLs was performed (FIG. 3D). A clear deviation in all the genotypes towards the effector/effector memory population was observed when compared to the CRE controls (FIGS. 3D and 3E). For comparison purposes the effector/effector memory window population was expanded to cover a wide range of CD44 expression, nevertheless the dKO cells tended to lose the expression of this receptor as is shown by comparing MFI values of that axis (FIG. 3E).

### Example 4

### Inhibition of TCPTP And PTP1B With Pharmacological Inhibitors

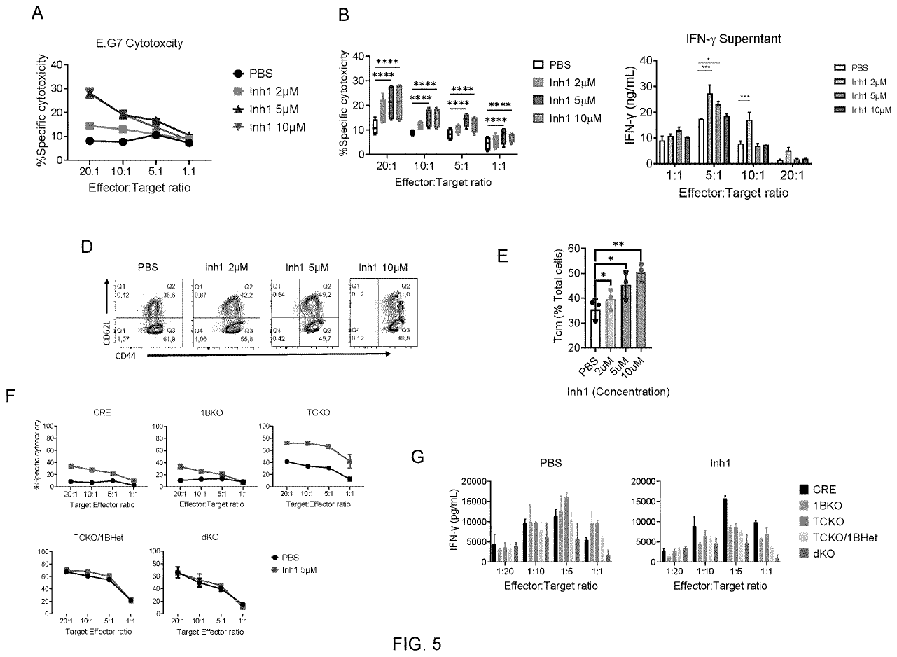

Inhibition of both enzymes resulted in enhanced in vitro functionality of CD8 T cells, an effect with potential benefits to reverse inhibitory cues in tumor microenvironments. Next, small molecule inhibitors targeting specifically TC-PTP and PTP1B were tested, taking advantage of their similarities in their catalytic domain [36]. L598 (from here on called Inh1) is a competitive orthosteric soluble compound that can inhibit both phosphatases with equivalent IC₅₀ [37]. Given that the main known substrates of TCPTP and PTP1B are transcription factors or signaling components lying close upstream, the functional changes observed in genetic deficient were assumed to require modulation of gene expression. Hence, OT-1 CTLs were generated in vitro in presence of various concentrations (2, 5 and 10 μM) of Inh,1 and they were tested at day 5 in cytotoxic assays against E.G7 targets (FIGS. 5A and 5B). Treatment with the compound significantly increased the cytotoxic activity of CTLs against E.G7 targets at all concentrations (FIGS. 5A and 5B). The cytotoxic activity was highest at 5 μM, and no significant differences between 5 μM and 10 μM was observed, uncovering a limited effect for the inhibitor. As cytokine secretion seems to be also affected by the reduced activity of TC-PTP and PTP1B IFN-γ secretion was quantified in the supernatant of these experiments by ELISA (FIG. 5C). As observed in genetic deficient cells, IFN-γ secretion was higher in the lower effector to target ratios, suggesting that low levels of stimulation favor proinflammatory cytokine secretion. In addition, presence of the inhibitor at low concentration (2 μM) correlated with a more significant increase in the secretion of IFN-γ, resembling observations in the single TC-PTP or PTP1B deficient cells. Additionally, increasing the concentration of the inhibitor led to an opposite effect on IFN-γ secretion, similarly to that observed in the TCKO/1 BHet or the dKO derived CTLs.

As the differentiation state of CD8 T cells was altered in the various genotypes analyzed before, how treatment with Inh1 altered the expression of differentiation markers was determined by flow cytometric analysis (FIGS. 5D and 5E). Expression of CD62L and CD44 drifted towards a gradual increase of the Tcm population (CD62L high, CD44 high events) which correlated to the inhibitor concentration used (FIG. 5E), a marker associated with effector differentiation, correlated to the inhibitor concentration used. The observations showed that treatment with Inh1 reproduced the phenotypic consequences of combined TC-PTP and PTP1B deficiency in CD8 T cells. To test if the phenotype observed after treatment with Inh1 is a consequence of specific inhibition of TCPTP and PTP1B, CD8 T cell of the different the genotypes used previously were treated with 5 μM Inh1 during in vitro CTL differentiation and tested for specific cytotoxicity against E.G7 cells (FIG. 3F). As seen in WT OT-1 CTLs, CRE controls increased their capacity to lyse E.G7 target cells about 2-fold. In the case of PTP1B single deficient cells, cytotoxicity followed a pattern without significant differences to CRE controls, either when treated or not with the inhibitor. Remarkably, PTP1B deficient CTLs treated with the inhibitor increased their cytolytic capacity to that of TCPTP single deficient cells. These results suggest a requirement for primary TCPTP inhibition for the enhanced activity observed in CD8 cells, and the ability of the inhibitor to target TCPTP activity intracellularly. However, after treatment with the inhibitor, TCPTP single deficient cells still increased their cytolytic activity twice as much when compared to non-treated cells. This increase resembles the increase observed when comparing the TCPTP KO with TCKO/1BHet CTLs. Hence, PTP1B is targeted intracellularly by the inhibitor and the pharmacological inhibition with 5 μM Inh1 has a comparable effect to the observations in the PTP1B hemizygous cells. Remarkably, the inhibitor did not have an effect on either TCKO/1 BHet or dKO CTLs cytotoxic capacities, suggesting that in both of these genotypes, cells are brought to their maximum effector capabilities (FIG. 5F). Next, IFN-γ was measured in the cell supernatant of these experiments. As observed previously, cells stimulated at 5 to 1 effector to target ratio in either genetically deficient in TCPTP or those treated with the inhibitor had the highest IFN-γ secretion (FIG. 5G). In general, as seen in the previous experiments, reduced activity of TCPTP and PTP1B led to a decrease in the secretion of IFN-γ.

### Example 5

### Inhibition of TCPTP and PTP1B With Pharmacological Inhibitors

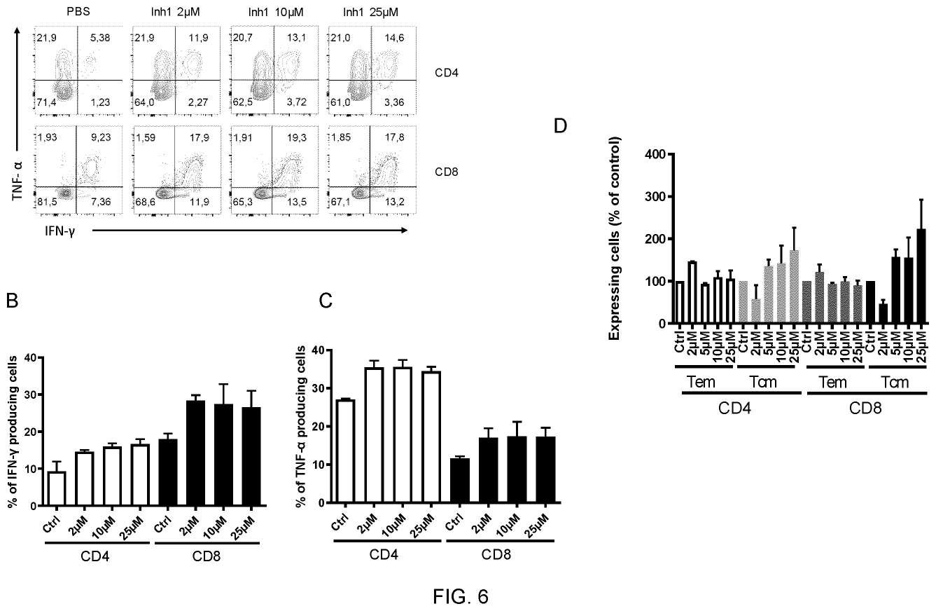

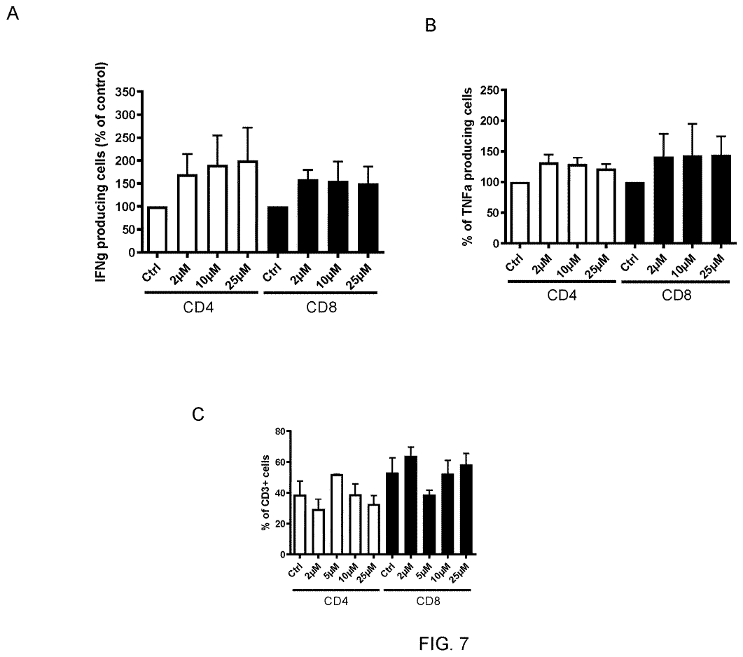

Next, the capacity of Inh1 to modify the functionality of human T cells was teste. To do so, T cells were enriched from healthy donors PBMCs and stimulated in vitro with anti-CD3 and anti CD28 for 7 days in the presence of increasing amounts of Inh1. At day 7 the cells were harvested and analyzed by surface and intracellular flow cytometry for the proinflammatory cytokines IFN-γ and TNF-α, and for the differentiation markers CD45RO and CD62L defining effector and memory populations. In vitro treatment of human T cells with Inh1 led to an increase in the production and accumulation of both IFN-γ and TNF-α in both CD4 and CD8 T cells (FIGS. 6A, 6B, 7A and 7B). In all cases the increase seemed to reach its maximum with 2 μM treatment and be maintained at similar extent when treated with increased concentrations. Increase in cytokine production was around 30 to 40% for TNF-α and 50 to 70% for IFN-γ, in both CD4 and CD8 T cells. Besides increased proinflammatory cytokine production, also it was also observed that treatment with Inh1 skewed T cell differentiation towards a central memory phenotype (CD45RO high, CD62L high) in both CD4 and CD8 T cells (FIG. 6C). This increase was more pronounced in CD8 T cells and seemed to correlate to the concentration of inh1 used. We did not find any preference on improving CD4 or CD8 T cells proliferation as both cell types were similarly represented at the different concentrations used of Inh1 (FIG. 7C).

### Example 6

### Bulk mRNA Sequencing

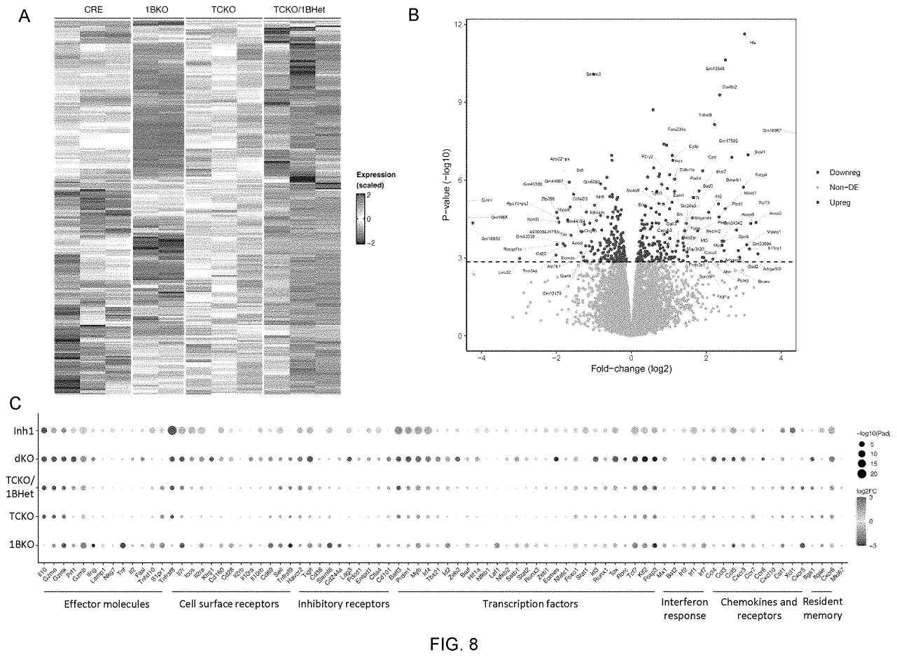

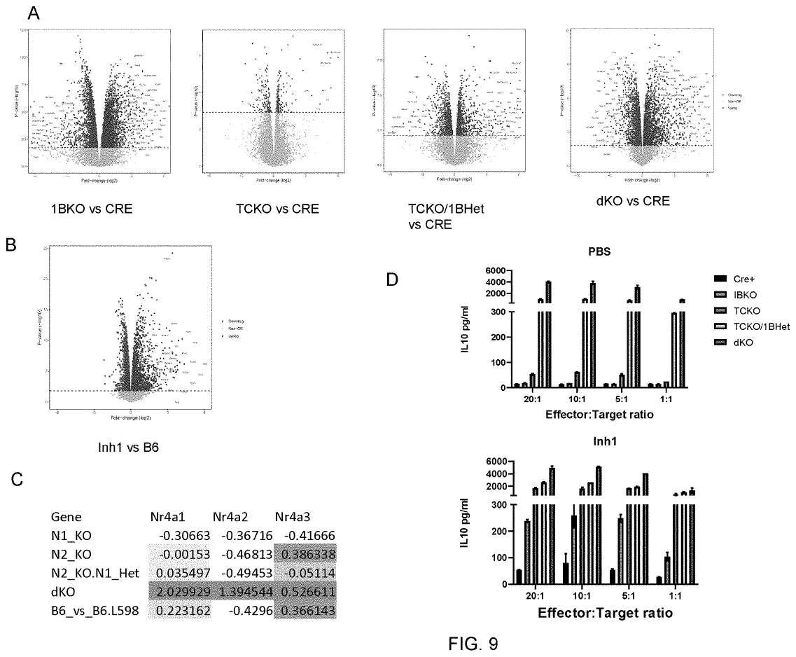

TCPTP and PTP1B activity regulate several pathways that control transcriptional activity. To understand the changes in gene expression that led to an increase in the effector qualities of CD8 T cells with decreased activity of TCPTP and PTP1B bulk mRNA sequencing of CTL derived from the different genotypes as well as from cells treated with Inh1 was performed. The aim was to identify the differentially regulated transcripts by comparative RNAseq (FIGS. 8 and 9). To eliminate bias given by the changes in peripheral CD8 differentiation observed in vivo, we generated CTLs were generated from purified naïve splenic CD8 T cells. The most significantly differentially expressed genes were observed when comparing 1 BKO and dKO samples to CRE controls (FIG. 9A). The interest was centered on differences between the TCPTP KO cells and the PTP1B Het/TCPTP KO cells, to determine the genes implicated in the enhanced effector properties. A heatmap built on the differential expression between TCKO/1 BHet and CRE controls showed a clear resemblance with the genetic footprint of the single TCKO (FIG. 8A). However, the single 1 BKO footprint distributed similarly to that of CRE controls, highlighting firstly divergent regulatory functions between both genes, and secondly the role of TCPTP in controlling genes related to immune response. Still, some downregulated and upregulated transcripts coincided between the 1 BKO samples and the TCKO/1BHet. At the same time, their expression levels were different than that of the TCKO implying that some PTP1B regulated genes contribute to the phenotype of the TCKO/1BHet cells (FIG. 8A). To highlight these differences, the transcriptomes of TCKO/1 BHet cells were directly compared to the TCKO cells (FIG. 8B). 376 differentially expressed genes were identified (Table 1 presents genes showing ±0.5 log₂ fold change). This list of differentially expressed genes shows a transcriptome that leans towards an effector T cell phenotype, characterized by positively regulated effector genes such as Prf1, and Gzmb, potentially as consequence of the downregulation of the transcription factors Tcf7 and EOMES [38], and downregulation of the naïve/memory marker Sell (CD62L). It was also noticed that the triggering of counter-modulatory mechanisms, as the inhibitory receptor klrk1, the Jak/STAT regulator ASB2 and the cytokines IL-10, Tgfb3 and cytokine receptor for IL-13, IL-13ra1, were upregulated, potentially by the presence of the transcription factor Maf, which was also found increased. Given the large amount of data generated, a biased approach was used to highlight the transcriptomic signature of the different genotypes analyzed based on known CD8 T cell functionally relevant genes. A comparative signature between CTL differentiated in vitro from C57BL/6 naïve spleen CD8 T cells treated or not with 5 μM Inh1 (FIG. 5C) was added to this analysis. As the results presented above suggest, TCPTP deficiency corresponds to an increase in effector molecule expression (e.g., granzymes and perforin), reduction of migratory receptors to secondary immune organs (e.g., CD62L and CCR7), mild expression of inhibitory receptors (e.g., PD1, Tigit and Tim-3), and increased expression of chemokines CCL3, 4 and 5. Surprisingly, deletion of PTP1B potentiated this effector phenotype in the TCPTP deficient background, an effect that correlated to the number of alleles deleted, been more evident in the dKO than in the TCKO/1 BHet cells. In contrast, there was a large discrepancy for the transcriptomic signature of the PTP1B deficient cells when compared to all other cells analyzed. 1 BKO cells transcriptome resembled that of memory T cells, characterized by the upregulation of the IL-7 receptor CD127, CD62L, CXCR5 and CD28, and upregulation of the transcription factor Tcf1. These cells also showed characteristics of exhausted T cells like high expression of the inhibitory receptors Tigit, SLAMF6, CD38 and CTLA4. Similarly, although the transcriptome of dKO cells correlated to that of single TCPTP KO cells, these dKO cells also expressed higher levels of receptors associated with exhaustion, as Tim-3, Tigit, Lag3, Entpd1 and PD-1. Even more, ectopic expression of the Th17 related transcription factor Rorc in dKO suggests dysregulation of differentiation, a phenomenon observed in CD4 T_{reg} cells [39]. Supporting even further the dysfunctional state of dKO cells, upregulation of the three members of the transcription factor family NR4A (FIG. 9C) associated with establishment of exhaustion in CD8 T cells was detected [40]. These observations are in line with the phenotypical and functional analyses discussed above, supporting the idea that complete inhibition of both of the TCPTP and PTP1B enzymes leads to important developmental dysregulation. Correspondingly, the transcriptome of CD8 T cells treated with Inh1 highly resembles that of the TCKO and TCKO/1 BHet and confirming that the mechanisms improving the effector qualities of T cells treated with Inh1 are similar to the changes observed in the genetically deficient T cells. IL-10 is one of the highly upregulated mRNA species in the TCKO, TCKO/1BHet, dKO and the T cells treated with the inh1. Traditionally considered as a regulatory cytokine, IL-10 has been more recently associated to improved antitumoral responses and prevention of CD8 T cell exhaustion. The presence of IL-10 in the supernatants of in vitro cytotoxicity assays was tested by ELISA (FIG. 9D). IL-10 secretion in these conditions followed an almost identical pattern to the findings in cytotoxicity discussed above. PTP1B single deficient T cells were no different than CRE controls. In contrast TCKO, TCKO/1 BHet and dKO cells showed a gradual increment of IL-10 secretion in their corresponding supernatants. Furthermore, additional treatment with 5 μM Inh1 resulted in an almost global increase in IL-10 secretion on the conditions tested except for the T cells derived from dKO mice, which were insensitive to the presence of Inh1. As an alternative readout to cytotoxicity experiments, secretion of IL-10 confirmed the effects of gradual inhibition of these enzymes and the specificity of the inhibitor.

TABLE 1
Differentially expressed genes showing ±0.5 log₂ fold change

| Upregulated Genes | | | Downregulated Genes | | |
|---|---|---|---|---|---|
| Gene | Log2FC | −LOG10 Pv | Gene | Log2FC | −LOG10 Pv |
| Gm16867 | 4.583613765 | 7.743969558 | Gas2l3 | −0.502012194 | 2.980671302 |
| Arhgef10l | 3.375523703 | 3.162380839 | Acss1 | −0.506466047 | 4.573504106 |
| Anxa3 | 3.323887376 | 4.370440005 | Nktr | −0.507405419 | 5.347222359 |
| Trp73 | 3.183864169 | 4.329619822 | Bicra | −0.510591592 | 5.445172534 |
| Il13ra1 | 3.148069558 | 3.347919405 | Hemgn | −0.51127167 | 4.547699236 |
| Sval1 | 3.113378248 | 6.97800749 | Tle4 | −0.520268744 | 6.7620124 |
| Maats1 | 3.073868754 | 3.509705509 | Sesn1 | −0.524984258 | 4.012194644 |
| Hlx | 3.017016953 | 11.62988197 | Prex1 | −0.528346061 | 6.951378128 |
| Fabp4 | 2.990486107 | 5.730548812 | Ccdc50 | −0.537479406 | 3.905213962 |
| Gad2 | 2.884878671 | 3.033409812 | Ash1l | −0.540294946 | 3.080580855 |
| Gm33994 | 2.85130388 | 3.282265842 | Tnfrsf26 | −0.542198363 | 3.33295241 |
| Akap6 | 2.805937074 | 4.076282813 | 2310043P16Rik | −0.549594177 | 4.31760093 |
| Gm17590 | 2.677061312 | 6.880135096 | Gata3 | −0.558759021 | 2.969691432 |
| Gm13546 | 2.511571834 | 10.62700473 | Tcf7 | −0.565319484 | 3.607971546 |
| Moxd1 | 2.505851231 | 4.898137715 | 2610005L07Rik | −0.566123816 | 3.339881344 |
| Arhgap6 | 2.475686655 | 3.287223596 | Evl | −0.583454136 | 5.369978764 |
| Gpx8 | 2.461602554 | 3.651273782 | Gm3200 | −0.593380172 | 2.87093051 |
| Gm34342 | 2.421787499 | 3.649760992 | Dync1h1 | −0.597933278 | 3.585904025 |
| Mycl | 2.42133903 | 3.496987024 | Cd27 | −0.600331588 | 3.486227603 |
| Bcam | 2.401218391 | 2.876069905 | Skp2 | −0.604852641 | 3.720076116 |
| Cox6b2 | 2.358188136 | 9.28164924 | Ehd3 | −0.608487427 | 3.245810206 |
| Plcd1 | 2.338691483 | 4.580231142 | Fam117a | −0.609620031 | 3.65232734 |
| Bmerb1 | 2.325565928 | 5.090164095 | Tmem154 | −0.626271396 | 3.228257557 |
| Tnfrsf8 | 2.21959723 | 8.143064317 | Lca5 | −0.629018682 | 3.0906724 |
| Ahrr | 2.171479872 | 2.958603479 | Elovl7 | −0.635050232 | 4.578446986 |
| Il10 | 2.065970066 | 4.763881612 | Tbxa2r | −0.637309936 | 2.931944483 |
| Kif1a | 1.978605223 | 2.893025761 | G2e3 | −0.655659246 | 6.102781899 |
| Pparg | 1.931965146 | 3.015266143 | Edaradd | −0.656367868 | 3.607642471 |
| Cpd | 1.902139606 | 6.356393186 | Zfp712 | −0.657208152 | 3.450530626 |
| Cass4 | 1.896038073 | 3.027369987 | Zfp831 | −0.661319463 | 3.655008638 |
| Ifitm7 | 1.808039402 | 5.578813721 | Ripor2 | −0.673148863 | 3.669836684 |
| Penk | 1.767763324 | 4.047145185 | Plxdc2 | −0.696129569 | 5.680476542 |
| Padi4 | 1.649125986 | 5.329849139 | Lrig1 | −0.699182853 | 3.096745954 |
| Batf3 | 1.630569268 | 4.543886131 | Sfmbt2 | −0.709083502 | 2.969245871 |
| Gm5111 | 1.529710663 | 2.835285989 | C330011M18Rik | −0.721230998 | 3.352165289 |
| Fndc3c1 | 1.501048629 | 3.070623856 | 2610035D17Rik | −0.723291251 | 3.015218775 |
| Map3k20 | 1.486652588 | 3.231948298 | Vps37b | −0.724687709 | 3.751745276 |
| Il7r | 1.465371972 | 4.335839407 | Prr5l | −0.726477405 | 3.056822748 |
| Slc24a3 | 1.41855724 | 4.047935999 | Klf2 | −0.745458141 | 3.218615173 |
| Nectin2 | 1.401208294 | 3.446159076 | Tmem71 | −0.77367078 | 3.19317443 |
| Mt3 | 1.40120788 | 3.305588471 | Pygm | −0.79206733 | 4.074225861 |

### Example 7

### Cytotoxic T Lymphocyte Stimulation with INF-γ

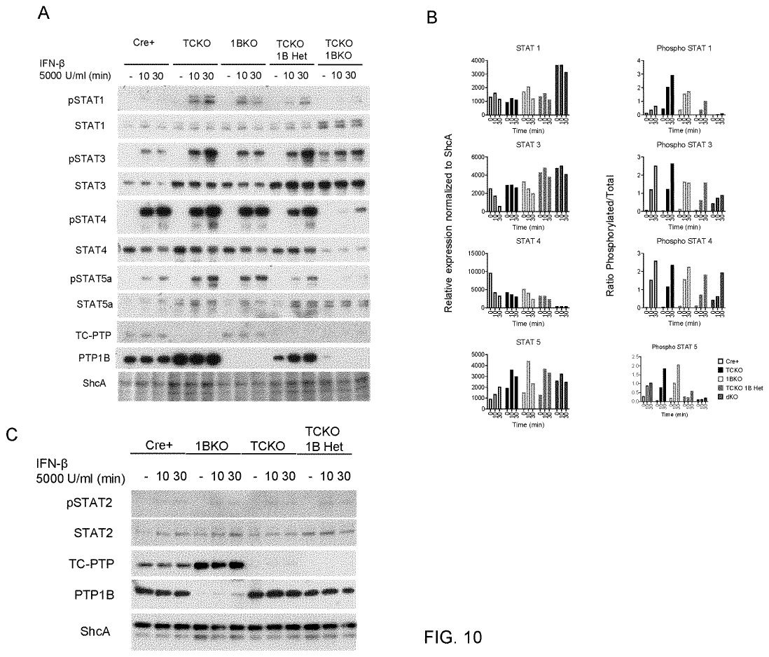

Previous reports on TCPTP deficient CD8 T cells identified dysregulation of Type I interferon signals as the main source for phenotypical changes. However, in the present analysis a strong dysregulation of interferon regulated genes was not observed in samples from the TCKO, TCKO/1BHet or dKO T cells. Indeed, upregulation of such mRNAs was only observed in samples from the 1 BKO animals. Type I interferon signals are transduced by members of the JAK and STAT families, which are well known targets of TCPTP and PTP1B. To investigate how the combined absence of these phosphatases modify Type I interferon signals, CTL carrying different genotypes were stimulated with IFN-γ for 10 and 30 minutes and the levels phosphorylation were analyzed on the canonical dimerizing c-terminal phosphotyrosine residues by immunoblotting with specific antibodies. CRE control cells responded to IFN-γ by increasing phosphorylation of these residues in STATs 1, 2, 3, 4 and 5a (FIGS. 10A, 10B and 10C) revealing the complexity of type I interferon signals in CD8 cells. No increase in the phosphorylation of STAT6 was observed either in CRE control cells or in any of the genotypes analyzed. Deficiency of either of these phosphatases resulted in the increased phosphorylation of STATS 1, 3 and 5, while phosphorylation levels of STAT4 seemed unaffected. A similar response pattern was observed in cells from TCKO/1 BHet, confirming that these cells are more sensitive to stimulation with type I interferons. Conversely, dKO cells were highly hyporesponsive with decreased phosphorylation of STATS 1, 3 and 5, and although the ratio of phosphorylated STAT4 to total protein was conserved, a clear restriction in protein expression in these cells resulted in low availability of the phosphorylated version. dKO cells were largely different to controls at basal level as well, where protein expression and levels of phosphorylation were also affected. Particularly, and in opposition to STAT4, increased expression of STATs 1 and 3 was detected, accompanied with increased basal level phosphorylation of STAT3. Higher expression and basal phosphorylation of STAT3 was also seen in TCKO and TCKO/1 BHet samples, potentially related to the global changes in transcription seen in these cells.

### Example 8

### Knock Down of Transcription Factor BatF3

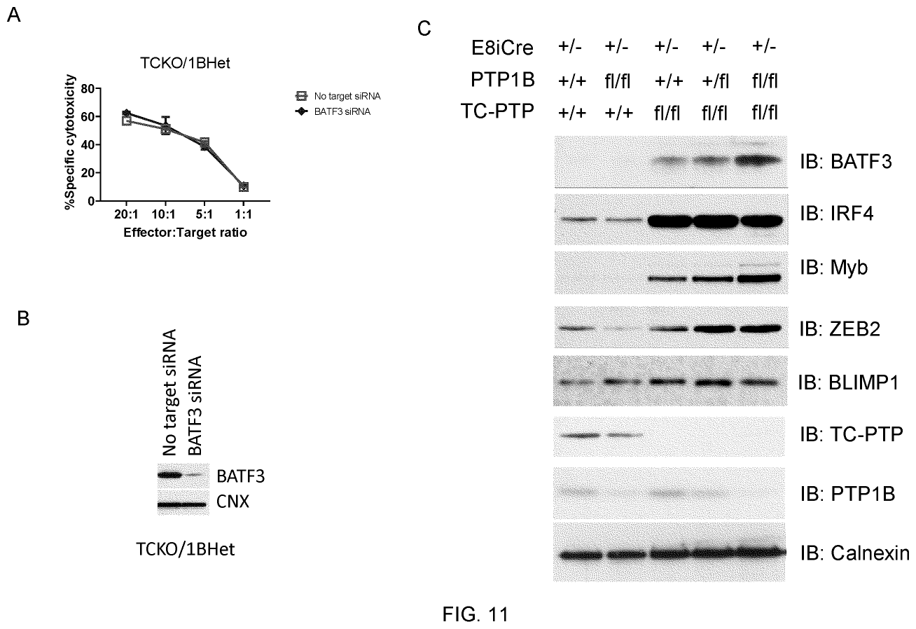

One of the potential downstream mechanism by which TCPTP/PTP1B deficiency takes effect is the upregulation of transcription factor BatF3. BatF3 is linked to the differentiation of CD8+ dendritic cells into a phenotype with enhanced capacities to activate CD8+ T cells, named conventional DC type I (cDC1). To test a possible link between BatF3 and the enhanced CD8+ cytotoxicity as a potential master transcription factor driving the enhanced effector properties of TCKO/1 BHet cells, the expression of was knocked down the expression of BatF3 using an siRNA during the in vitro differentiation of TCKO/1BHet CTLs under the OT-1 background. Using the EG.7 cytotoxicity assay as readout, the functional effector properties of TCKO/1 BHet cells with reduced expression of BatF3 were compared (FIG. 11A). No appreciable differences in their cytotoxic ability were observed against E.G7 cells when compared to controls transfected with a non-targeting siRNA, despite a clear reduction of BatF3 expression (FIG. 11B). Even though BatF3 was the most significantly upregulated transcription factor observed in the analysis, the expression of transcripts of other transcription factors know to positively drive CD8 effector differentiation were also increased in those genotypes showing an enhanced effector genotype (FIG. 11C). Immunoblotting confirmed an elevation of protein products for IRF4, Myb, ZEB and BLIMP1, suggesting that transcriptomic changes observed between the different genotypes has a multifactorial origin.

### Example 9

### In Vivo Treatment with Inhibitor 1

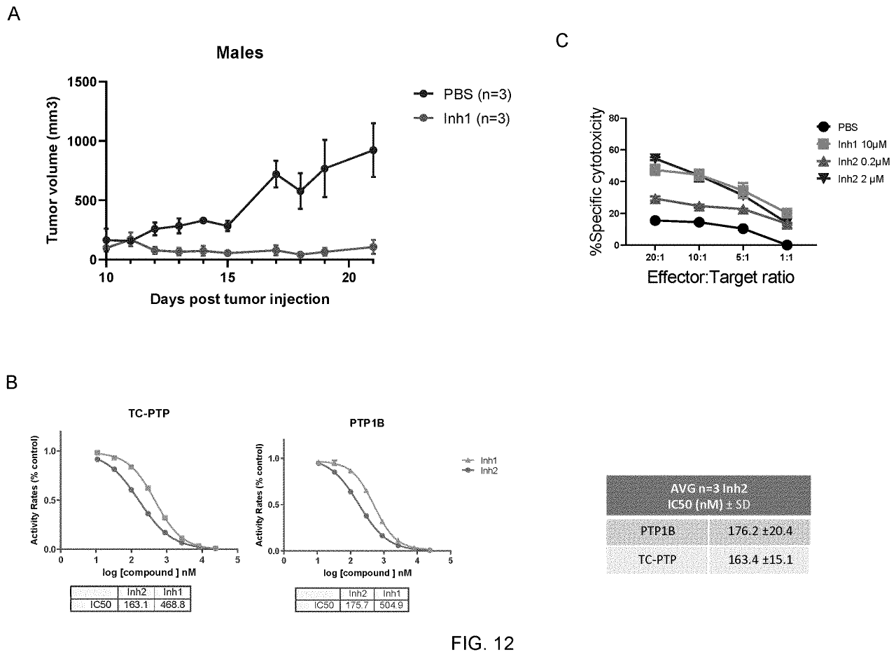

TCPTP and PTP1B have been associated with the control of proinflammatory signals not only in T cells, but also in other immune and non-immune cells. Indeed, it has been proposed that decreased activity of these phosphatases may be beneficial to antitumoral responses. To test the antitumoral activity of Inh1 in vivo, the compound was injected in WT C57BL/6 mice bearing E.G7 thymoma. TCPTP and PTP1B are ubiquitous enzymes with a broad range of activities involving several cytokine and growth factor receptor signals transduction. To avoid systemic side effects resulting from the use of Inh1, the compound was injected intratumorally, limiting its activity to the tumor microenvironment. To do so, E.G7 thymoma tumors were injected in WT C57BL/6 mice and their growth was monitored by caliper measurement. Tumors were injected with Inh1 from day 10 every 48 hours with an equivalent of 50 μM of the compound or with PBS (FIG. 12A). As expected, in PBS injected controls, tumor growth proceeded exponentially. Conversely, tumors injected with Inh1 maintained a similar size during the extent of the observations. Treatment with Inh1 clearly and unexpectedly limited tumor growth, potentially through cells intrinsic and immune mechanisms.

### Example 10

### Combined In Vivo Treatment with Inhibitor 2 And Anti PD-1 Therapy

As EG.7 cells are engineered to express the exogenous antigen OVA, they can artificially contribute to the capacity of Inh1 to induce a targeting immune response. To avoid such potential artifact, the syngeneic melanoma model B16F10 was used in the C57BL/6 background. The syngeneic melanoma model B16F10 is a tumor model known to be poorly immunogenic. In these experiments, an alternative molecule from the same series of compounds as Inh1, here in called Inhibitor 2 (Inh2), was used. Inh2 is disclosed in WO2008089581 and the following formula:

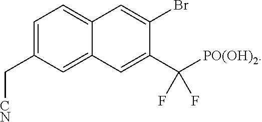

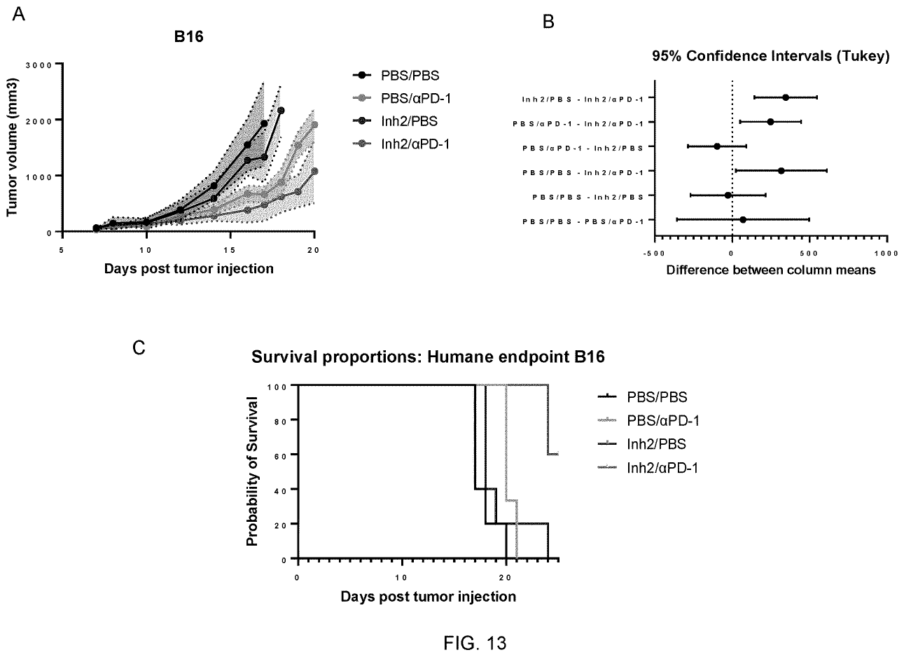

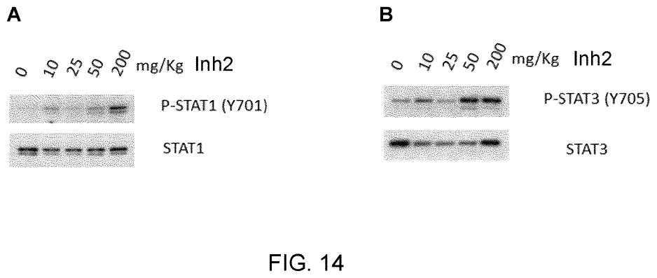

This compound has been used in phase 1 clinical studies, and it is known to be tolerated systemically in mice and humans. Inh2 has more potent inhibitory capacity, with an IC₅₀ in vitro up to 3 times higher for both enzymes (FIG. 12B) which was reflected in its capacity to induce similar biological activity to Inh1 at concentrations 5 times lower (FIG. 12C). B16F10 melanoma is a “cold tumor” cancer model used to demonstrate the immunogenetic properties of immune checkpoint blockade therapies, including those based in anti-PD-1 antibodies with important clinical success. To test the capacity of TCPTP and PTP1B inhibition combined with immune checkpoint blockade therapies, to limit B16F10 tumors growth, the compound and an anti-PD-1 monoclonal antibody were injected intratumorally as described above. The anti-PD-1 antibody used is the RMP1-14 monoclonal antibody which reacts with mouse PD-1 (also known as CD279) from BioXCell, Cat. No. BP0146. It is a rat IgG2a, K. Now referring to FIG. 13. Although treatment with the Inh2 alone slowed tumoral growth, its effect was not significant (FIGS. 13A and B). Conversely, single treatment with anti-PD-1 showed a significant reduction of tumor growth and prolonged survival, as reported before. Unexpectedly, the combined use of anti-PD1 and Inh2 significantly further reduced the development of the B16F10 tumor when compared to anti-PD-1 therapy alone. Furthermore, combined therapy resulted in 100% subject survival at day 25 post injection.

### Example 11

### Discussion

The present application shows the combined influence of the phosphatases PTP1B and TCPTP in CD8 T cell function, and their potential as immunotherapeutic targets. Herein, the effects of reduced expression of both genes in CD8T cells was studied. Double deleted mutant cells displayed a heavily modified phenotype resembling terminally differentiated effector T cells. These T cells made up to 80% of peripheral CD8 T cells in young unchallenged mice, suggesting that they were spontaneously activated, since control CRE expressing animals displayed only about 10% terminally differentiated effector T cells. Functional analysis supports the phenotypical findings as these T cells were highly cytotoxic against antigen specific target cells. Deeper study of their differentiation state at protein and mRNA level placed dKO cells as terminal exhausted cells with high expression of Tim-3, TIGIT, LAG-3, CD244a, PD1 and Entpd1. These cells also highly expressed all the members of the NR4A family of transcription factors, known to mediated CD8 exhaustion [40] and showed altered STAT transcription factors expression.

Regarding the single deficiency of TCPTP in CD8 T cells, the results above show that it confers better effector T cell qualities against tumors while providing protection from T cell exhaustion [20, 21]. Surprisingly, when TCPTO deficiency is accompanied by partial deletion of PTP1B, e.g., in TCKO/1BHet T cells, the phenotype of TCPTP deficient cells was potentiated with an even greater increased in the cytolytic ability on antigen specific target cells. Nevertheless, these TCKO/1 BHet T cells did not show the extreme phenotypical or transcriptional changes observed in the double deficient T cells. Single PTP1B deficient CD8 T cells were highly divergent from TCPTP deficient cells. Surprisingly, the transcriptomic signature of TCKO/1 BHet and dKO T cells nevertheless resembled accurately that of the TCPTP single deficient cells. PTP1B single deficient T cells phenotypically did not resemble effector CD8 T cells, nor did they show improved functional effector qualities over CRE expressing controls. These findings are contracting with those from others [22]. One potential caveat in Wiede's et al study [22] is the use of CRE negative controls, leaving unaccounted potential nonspecific consequences of CRE expression [33, 34].

A direct correlation of the cytotoxic capacity of CD8 T cells to the degree of functional silencing of TCPTP and PTP1B was observed, either genetically or pharmacologically, corresponding to the differentiation stage. However, secretion of IFN-γ did not follow such correlation. Instead, a peak production with mild inhibition was observed, dictated either by low or intermediate concentrations of inh1 or by deleting each phosphatase gene individually. However, at higher levels of inhibition (either uncombined or from combined genetic/pharmacological inhibition) production of IFN-γ decreased. This discrepancy between readouts of effector function is interpreted as resulting from control by independent feedback mechanisms, the understanding of which will broaden the range for future therapeutic intervention. Conversely, as predicted but mRNA analysis, IL-10 secretion correlated with the cytotoxic capacity of the T cells and with the level of inhibition of TCPTP/PTP1B. IL-10 is classically regarded as an immunosuppressive cytokine, however recent literature highlights a role for this cytokine in reprograming metabolically exhausted CD8 T cells favoring antitumor responses [1], a potential mechanism involved in the enhanced antitumoral effects of TCPTP/PTP1B inhibitors. Autocrine stimulation by the secreted IL-10 can also explain the increase of phosphorylated STAT3 seen after type I IFN stimulation of TCPTP/PTP1B deficient cells, further reinforced by elevated expression of STAT3. The secretion pattern of IL-10 in the different cells analyzed suggests a commanding regulatory role for TCPTP in T cells as its deletion was required to observe an effect from PTP1B inhibition/deletion. This opposes previous findings in macrophages [42], where PTP1B deficiency alone caused increase of IL-10 production. The proinflammatory phenotype observed in TCPTP deficient cells has been explained by its regulatory role on IFN pathways, more specifically for its roles as a nuclear and cytoplasmic phosphatase for JAK1 and STAT1 [6, 19, 25]. However, the data presented herein does not support dysregulation of IFN signals as a potential mechanism given that TCPTP/PTP1B deficient cells experienced a reduction rather than increase of interferon response related genes.

Finally, the presented data offers a mechanistic insight on the advantages of developing dual TCPTP/PTP1B specific inhibitors in immunotherapeutic settings. The high homology between both catalytic domains provides a foundation to prevent of off-target consequences. Given its transductional independence from other T cell checkpoint pathways, combinatorial use of TCPTP/PTP1B inhibitors with already immune checkpoint inhibition therapies is an unexpected and surprising new therapeutic avenue. Indeed, synergy of TCPTP/PTP1B inhibitors with an anti-PD-1 blockade model is shown herein. Furthermore, TCPTP/PTP1B inhibition does not have to be T cells specific as effects in tumoral [19] and other immune components, as dendritic cells [7], promotes further antitumoral activity.

### Example 12

### Stat Phosphorylation in PBMC of C57BL/6 Mice after 2 Hours of Intraperitoneal Injection of INH₂

Protein lysates from PBMC extracted from mice treated with 25 mg/kg, 50 mg/kg, 100 mg/kg and 200 mg/kg of Inh2. Samples were migrated in 10% SDS-PAGE gels followed by transfer to PVDF membranes and immunoblot. 10 μg/lane of protein sample loaded. Blot A: anti-Phospho-STAT1 (top), stripped and re-blotted with anti-STAT1 (bottom). Blot B: anti-Phospho-STAT3, stripped and re-blotted with anti-STAT3.

These results demonstrate the capacity of Inh2 to increase the signaling of JAK/STAT pathway, specifically the tyrosine phosphorylation of STAT1 and STAT3 proteins, in blood immune cells of mice after systemic injection. These results provide evidence supporting the effects of the drugs of the present invention on the expected cellular targets and provide supporting evidence for a method to monitor on the activity of the compound in patients and adjust dosage as may be necessary during treatment. In the present example, monitoring is performed with immunoblotting, but other means of monitoring such as fluorescence analysis with fluorescence activated cell sorting or like methods could be envisioned.

### REFERENCES

1. Hashimoto, M., et al., CD8 T Cell Exhaustion in Chronic Infection and Cancer: Opportunities for Interventions. Annual Review of Medicine, 2018. 69(1): p. 301-318.
2. Pike, K. A. and M. L. Tremblay, TC-PTP and PTP1B: Regulating JAK-STAT signaling, controlling lymphoid malignancies. Cytokine, 2016. 82: p. 52-7.
3. You-Ten, K. E., et al., Impaired Bone Marrow Microenvironment and Immune Function in T Cell Protein Tyrosine Phosphatase-deficient Mice. Journal of Experimental Medicine, 1997. 186(5): p. 683-693.
4. Heinonen, K. M., et al., T-cell protein tyrosine phosphatase deletion results in progressive systemic inflammatory disease. Blood, 2004. 103(9): p. 3457-3464.
5. Miller, B. C., et al., Subsets of exhausted CD8+ T cells differentially mediate tumor control and respond to checkpoint blockade. Nature Immunology, 2019. 20(3): p. 326-336.
6. Heinonen, K. M., et al., Protein tyrosine phosphatases PTP-1B and TC-PTP play nonredundant roles in macrophage development and IFN-γ signaling. Proceedings of the National Academy of Sciences, 2009. 106(23): p. 9368-9372.
7. Penafuerte, C., et al., Downregulation of PTP1B and TC-PTP phosphatases potentiate dendritic cell-based immunotherapy through IL-12/IFNγ signaling. Oncolmmunology, 2017. 6(6): p. e1321185.
8. Galon, J., et al., Type, Density, and Location of Immune Cells Within Human Colorectal Tumors Predict Clinical Outcome. Science, 2006. 313(5795): p. 1960-1964.
9. Kmiecik, J., et al., Elevated CD3+ and CD8+ tumor-infiltrating immune cells correlate with prolonged survival in glioblastoma patients despite integrated immunosuppressive mechanisms in the tumor microenvironment and at the systemic level. Journal of Neuroimmunology, 2013. 264(1): p. 71-83.
10. Djenidi, F., et al., CD8<sup>+</sup>CD103<sup>+</sup>Tumor-Infiltrating Lymphocytes Are Tumor-Specific Tissue-Resident Memory T Cells and a Prognostic Factor for Survival in Lung Cancer Patients. The Journal of Immunology, 2015. 194(7): p. 3475-3486.
11. Dudley, M. E., et al., Cancer Regression and Autoimmunity in Patients After Clonal Repopulation with Antitumor Lymphocytes. Science, 2002. 298(5594): p. 850-854.
12. Berger, C., et al., Adoptive transfer of effector CD8+ T cells derived from central memory cells establishes persistent T cell memory in primates. The Journal of Clinical Investigation, 2008. 118(1): p. 294-305.
13. Rosenberg, S. A., et al., Durable Complete Responses in Heavily Pretreated Patients with Metastatic Melanoma Using T-Cell Transfer Immunotherapy. Clinical Cancer Research, 2011. 17(13): p. 4550-4557.
14. Haslam, A., J. Gill, and V. Prasad, Estimation of the Percentage of US Patients With Cancer Who Are Eligible for Immune Checkpoint Inhibitor Drugs. JAMA Network Open, 2020. 3(3): p. e200423-e200423.
15. Waldman, A. D., J. M. Fritz, and M. J. Lenardo, A guide to cancer immunotherapy: from T cell basic science to clinical practice. Nature Reviews Immunology, 2020. 20(11): p. 651-668.
16. Townsend, E. C., et al., The Public Repository of Xenografts Enables Discovery and Randomized Phase II-like Trials in Mice. Cancer Cell. 29(4): p. 574-586.
17. Penafuerte, C., et al., Mining the Complex Family of Protein Tyrosine Phosphatases for Checkpoint Regulators in Immunity. 2017, Springer Berlin Heidelberg: Berlin, Heidelberg. p. 1-24.
18. Simoncic, P. D., C. J. McGlade, and M. L. Tremblay, PTP1B and TC-PTP: novel roles in immune-cell signaling This paper is one of a selection of papers published in this Special issue, entitled Second Messengers and Phosphoproteins—12th International Conference. Canadian Journal of Physiology and Pharmacology, 2006. 84(7): p. 667-675.
19. Manguso, R. T., et al., In vivo CRISPR screening identifies Ptpn2 as a cancer immunotherapy target. Nature, 2017. 547: p. 413.
20. LaFleur, M. W., et al., PTPN2 regulates the generation of exhausted CD8+ T cell subpopulations and restrains tumor immunity. Nature Immunology, 2019. 20(10): p. 1335-1347.
21. Wiede, F., et al., PTPN2 phosphatase deletion in T cells promotes anti-tumour immunity and CAR T-cell efficacy in solid tumours. The EMBO Journal, 2019. n/a(n/a): p. e103637.
22. Wiede, F., et al., PTP1B Is an Intracellular Checkpoint that Limits T-cell and CAR T-cell Antitumor Immunity. Cancer Discovery, 2022. 12(3): p. 752-773.
23. Myers, M. P., et al., TYK2 and JAK2 Are Substrates of Protein-tyrosine Phosphatase 1B. Journal of Biological Chemistry, 2001. 276(51): p. 47771-47774.
24. Lu, X., et al., PTP1B is a negative regulator of interleukin 4-induced STAT6 signaling. Blood, 2008. 112(10): p. 4098-4108.
25. Simoncic, P. D., et al., The T cell protein tyrosine phosphatase is a negative regulator of janus family kinases 1 and 3. Curr Biol, 2002. 12(6): p. 446-53.
26. Bourdeau, A., et al., Inhibition of T Cell Protein Tyrosine Phosphatase Enhances Interleukin-18-Dependent Hematopoietic Stem Cell Expansion. STEM CELLS, 2013. 31(2): p. 293-304.
27. Doody, K. M., A. Bourdeau, and M. L. Tremblay, T-cell protein tyrosine phosphatase is a key regulator in immune cell signaling: lessons from the knockout mouse model and implications in human disease. Immunological Reviews, 2009. 228(1): p. 325-341.
28. Bourdeau, A., et al., TC-PTP-deficient bone marrow stromal cells fail to support normal B lymphopoiesis due to abnormal secretion of interferon-{gamma}. Blood, 2007. 109(10): p. 4220-8.
29. Klaman, L. D., et al., Increased Energy Expenditure, Decreased Adiposity, and Tissue-Specific Insulin Sensitivity in Protein-Tyrosine Phosphatase 1B-Deficient Mice. Molecular and Cellular Biology, 2000. 20(15): p. 5479-5489.
30. Bussières-Marmen, S., et al., Loss of T-cell protein tyrosine phosphatase in the intestinal epithelium promotes local inflammation by increasing colonic stem cell proliferation. Cellular & Molecular Immunology, 2018. 15(4): p. 367-376.
31. Zee, T., et al., T-Cell Protein Tyrosine Phosphatase Regulates Bone Resorption and Whole-Body Insulin Sensitivity through Its Expression in Osteoblasts. Molecular and Cellular Biology, 2012. 32(6): p. 1080-1088.
32. Maekawa, Y., et al., Notch2 integrates signaling by the transcription factors RBP-J and CREB1 to promote T cell cytotoxicity. Nature Immunology, 2008. 9: p. 1140.
33. Pépin, G., et al., Cre-dependent DNA recombination activates a STING-dependent innate immune response. Nucleic Acids Research, 2016. 44(11): p. 5356-5364.
34. Carow, B., et al., <em>lck</em>-Driven Cre Expression Alters T Cell Development in the Thymus and the Frequencies and Functions of Peripheral T Cell Subsets. The Journal of Immunology, 2016: p. 1600827.
35. Herndler-Brandstetter, D., et al., KLRG1+ Effector CD8+ T Cells Lose KLRG1, Differentiate into All Memory T Cell Lineages, and Convey Enhanced Protective Immunity. Immunity, 2018. 48(4): p. 716-729.e8.
36. Vidović, D. and S. C. Schürer, Knowledge-Based Characterization of Similarity Relationships in the Human Protein-Tyrosine Phosphatase Family for Rational Inhibitor Design. Journal of Medicinal Chemistry, 2009. 52(21): p. 6649-6659.
37. Montalibet, J., et al., Residues Distant from the Active Site Influence Protein-tyrosine Phosphatase 1B Inhibitor Binding. Journal of Biological Chemistry, 2006. 281(8): p. 5258-5266.
38. Danilo, M., et al., Suppression of Tcf1 by Inflammatory Cytokines Facilitates Effector CD8 T Cell Differentiation. Cell Reports, 2018. 22(8): p. 2107-2117.
39. Spalinger, M. R., et al., PTPN2 controls differentiation of CD4+ T cells and limits intestinal inflammation and intestinal dysbiosis. Mucosal Immunol, 2015. 8(4): p. 918-929.
40. Chen, J., et al., NR4A transcription factors limit CAR T cell function in solid tumours. Nature, 2019. 567(7749): p. 530-534.
41. Guo, Y., et al., Metabolic reprogramming of terminally exhausted CD8+ T cells by IL-10 enhances anti-tumor immunity. Nature Immunology, 2021. 22(6): p. 746-756.
42. Pike, K. A., et al., Protein Tyrosine Phosphatase 1B Is a Regulator of the Interleukin-10-Induced Transcriptional Program in Macrophages. Science Signaling, 2014. 7(324): p. ra43-ra43.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method for the treatment of cancer comprising administering to a subject in need thereof a therapeutically effective amount of a compound of structural Formula II, or pharmaceutically acceptable salts thereof, and stereoisomers thereof: 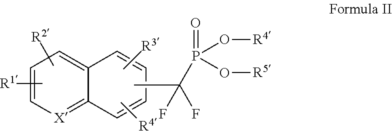
wherein X′ is selected from CH and N;
R^{1′} is selected from the group consisting of (a) C₁₋₃alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —CN, (e) —HC═NOH, (f) —(CH₃)C═NOH, (g) —HC═NOC₁₋₃alkyl optionally substituted with 1-3 halogens, (h) —(CH₃)C═NOC₁₋₃alkyl optionally substituted with 1-3 halogens (i) —C(═O)OC₁₋₃alkyl optionally substituted with 1-3 halogens, (j) —C(═O)NHR^{6′}, (k) —CH═CH-Phenyl wherein —CH═CH— is optionally substituted with 1-2 substituents independently selected from halogen and C₁₋₂alkyl optionally substituted with 1-3 F, (l) —CH₂CH₂-Phenyl wherein —CH₂CH₂— is optionally substituted with 1-4 substituents independently selected from halogen and C₁₋₂alkyl optionally substituted with 1-3 F, (m) Phenyl, (n) -HET-Phenyl, wherein HET is a 5- or 6-membered heteroaromatic ring containing 1-3 heteroatoms selected from O, N and S, (o) —C≡C-Phenyl, and (p) —CH₂-Phenyl, wherein the —CH₂— group of —CH₂-Phenyl is optionally substituted with 1-2 substituents independently selected from halogen and C₁₋₂alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH (iv) C1-3alkyl optionally substituted with 1-3 halogens, (v) —OC₁₋₃alkyl optionally substituted with 1-3 halogens, (vi) —SOₓMe, and (vii) —SO₂NH₂;
R^{2′} and R^{4′} are independently selected from H, halogen, —CH₃, —CF₃, —OCH₃, and —OCF₃;
R^{3′} is halogen, wherein said halogen is bonded to the fused aromatic ring of Formula II at a position ortho to the —CF₂PO(OR^{5′})₂ group,
R^{4′} and R^{5′} are each independently selected from the group consisting of:
(a) hydrogen;
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
(i) —(C═O)OR⁷,
(ii) —(C═O)NHR⁷,
(iii) —(C═O)N(R⁷)₂,
(iv) —(C═O)NH₂,
(v) —OR⁷,
(vi) —O(C═O)R⁷,
(vii) —O(C═O)OR⁷,
(viii) —O(C═O)NHR⁷,
(ix) —O(C═O)N(R⁷)₂,
(x) —O(C═O)NH₂,
(xi) —SO₂NH₂,
(xii) —SOₓCH₃,
(viii) —S(C═O)R⁷ and
(ix) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl; and
R^{6′} is selected from the group consisting of H, C₁₋₃alkyl optionally substituted with 1-3 halogens, Phenyl, and —CH₂-Phenyl, wherein Phenyl in both occurrences is optionally substituted with 1-3 substituents independently selected from (i) halogen, (ii) —C(═O)OC₁₋₃alkyl optionally substituted with 1-3 halogens, (iii) —C(═O)OH (iv) C₁₋₃alkyl optionally substituted with 1-3 halogens, and (v) —OC₁₋₃alkyl optionally substituted with 1-3 halogens;
R⁷ is selected from the group consisting of C₁₋₆ alkyl optionally substituted with 1-3 substituents independently selected from (i) halogen, (ii) hydroxy, (iii) —OC₁₋₃ alkyl, (iv) aryl, and (v) heteroaryl, wherein aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃alkyl;
R^{a} and R^{b} are each independently hydrogen or C₁₋₄ alkyl optionally substituted with hydroxy or 1-5 fluorines; and
x is 0, 1, or 2;
in combination with an immune checkpoint inhibitor.

2.-3. (canceled)

4. The method of claim 1, wherein said compound of formula II is of structural Formula IIa, or a pharmaceutically acceptable salts thereof, and stereoisomers thereof: 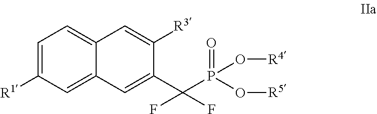
wherein
R^{1′} is selected from the group consisting of (a) C₁₋₃ alkyl optionally substituted with 1-3 halogens and optionally with one group selected from —OH, —OC₁₋₃alkyl optionally substituted with 1-3 halogens, —SOₓC₁₋₃alkyl, and —CN, (b) —C(═O)H, (c) —C(═O)C₁₋₃alkyl optionally substituted with 1-3 halogens, (d) —HC═NOH, (e) —(CH₃)C═NOH, (f) —HC═NOC₁₋₃alkyl optionally substituted with 1-3 halogens, (g) —(CH₃)C═NOC₁₋₃alkyl optionally substituted with 1-3 halogens, (h) —C(═O)OC₁₋₃alkyl optionally substituted with 1-3 halogens, (i) —C(═O)NHR^{6′}, (j) —CH═CH-Phenyl wherein —CH═CH— is optionally substituted with 1-2 substituents independently selected from halogen and C₁₋₂alkyl optionally substituted with 1-3 F, (k) —CH₂CH₂-Phenyl wherein —CH₂CH₂— is optionally substituted with 1-4 substituents independently selected from halogen and C₁₋₂ alkyl optionally substituted with 1-3 F, (1) Phenyl, (m) -HET-Phenyl, wherein HET is a 5- or 6-membered heteroaromatic ring containing 1-3 heteroatoms selected from O, N and S, (n) —C≡C-Phenyl, (o) —CH₂-Phenyl, and (p) —CN, wherein the —CH₂— group of —CH₂-Phenyl is optionally substituted with 1-2 substituents independently selected from halogen and C₁₋₂ alkyl optionally substituted with 1-3 F, wherein Phenyl and HET in all occurrences are optionally substituted with 1-3 substituents independently selected from (i) halogen, (ii) —C(═O)OC₁₋₃alkyl optionally substituted with 1-3 halogens, (iii) —C(═O)OH, (iv) C₁₋₃alkyl optionally substituted with 1-3 halogens, (v) —OC₁₋₃alkyl optionally substituted with 1-3 halogens, (vi) —SOₓMe, and (vii) —SO₂NH₂;
R^{3′} is halogen;
R⁴ and R⁵ are each independently selected from the group consisting of:
(a) hydrogen:
(b) aryl or heteroaryl wherein aryl and heteroaryl are optionally substituted with 1-3 halogens, C₁₋₃ alkyl, or C₁₋₃ haloalkyl; and
(c) —(CR^{a}R^{b})₁₋₂ substituted with one to two substituents independently selected from
(i) —(C═O)OR⁷,
(ii) —(C═O)NHR⁷,
(iii) —(C═O)N(R⁷)₂,
(iv) —(C═O)NH₂,
(v) —OR⁷,
(vi) —O(C═O)R⁷,
(vii) —O(C═O)OR⁷,
(viii) —O(C═O)NHR⁷,
(ix) —O(C═O)N(R⁷)₂,
(x) —O(C═O)NH₂,
(xi) —SO₂NH₂,
(xii) —SOₓCH₃,
(viii) —S(C═O)R⁷, and
(xiii) aryl or heteroaryl wherein aryl and heteroaryl are optionally substituted with 1-3 halogens, —CN, —SOₓCH₃, —SO₂NH₂, C₁₋₃ alkyl, C₁₋₃ haloalkyl, —OC₁₋₃ alkyl, or —OC₁₋₃ haloalkyl;
or R^{4′} and R^{5′} together with the phosphorus atom and the two oxygen atoms to which they are attached form a 5- to 7-membered ring optionally substituted with 1-3 substituents independently selected from (i) halogen, (ii) —(C═O)OC₁₋₃ alkyl, (iii) —(C═O)OH, (iv) C₁₋₃ alkyl optionally substituted with hydroxy or 1-3 halogens, (v) —OC₁₋₃ alkyl optionally substituted with 1-3 halogens, (vi) —OH, and (vii) aryl or heteroaryl wherein aryl and heteroaryl are optionally substituted with 1-3 halogens, C₁₋₃ alkyl, or C₁₋₃ haloalkyl;
R^{6′} is selected from the group consisting of H, C₁₋₃ alkyl optionally substituted with 1-3 halogens, phenyl, or —CH₂-phenyl, wherein phenyl is optionally substituted with 1-3 substituents independently selected from (i) halogen, (ii) —(C═O)OC₁₋₃ alkyl optionally substituted with 1-3 halogens, (iii) —COOH, (iv) C₁₋₃ alkyl optionally substituted with 1-3 halogens, and (v) —OC₁₋₃ alkyl optionally substituted with 1-3 halogens;
R⁷ is selected from the group consisting of C₁_alkyl optionally substituted with 1-3 substituents independently selected from (i) halogen, (ii) —OC₁₋₃ alkyl, (iii) aryl, and (iv) heteroaryl, wherein the aryl and heteroaryl are optionally substituted with 1-3 halogens, C₁₋₃ alkyl, C₁₋₃ haloalkyl, —CN, —SOₓCH₃, —SO₂NH₂, —COOH, and —OC₁₋₃ alkyl;
R^{a} and R^{b} are each independently hydrogen or C₁₋₄ alkyl optionally substituted with hydroxy or 1-5 fluorines; and
x is 0, 1, or 2.

5. The method of claim 4, wherein the compound is selected from the following compounds:

| Ex. | Structure |
|---|---|
| 2 | 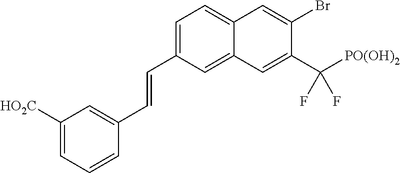 |
| 3 | 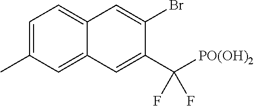 |
| 4 | 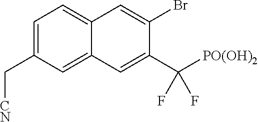 |
| 5 | 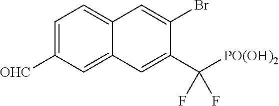 |
| 6 | 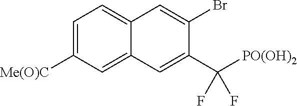 |
| 8a | 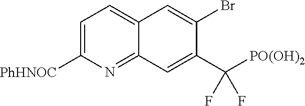 |
| 8b | 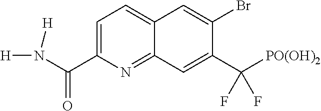 |
| 8c | 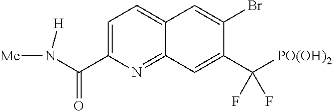 |
| 8d | 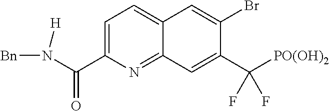 |
| 8e | 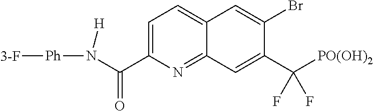 |
| 9a | 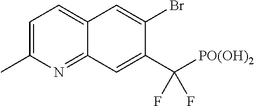 |
| 9c | 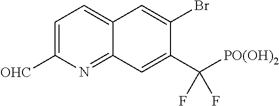 |
| 9d | 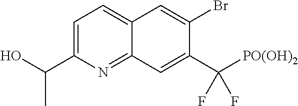 |
| 9e | 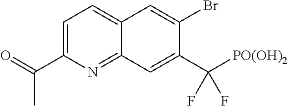 |
| 9f | 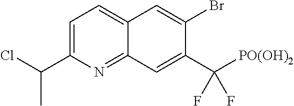 |
| 9g | 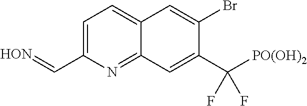 |
| 9h | 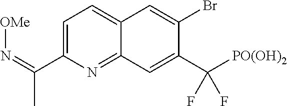 |
| L598 | 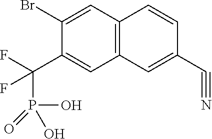 [SMILES: N#Cc1ccc2cc(Br)c(C(F)(F)P(=O)(O)O)cc2c1] |
| 10 | 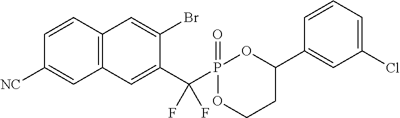 [SMILES: N#Cc1ccc2cc(Br)c(C(F)(F)P3(=O)OCCC(c4cccc(Cl)c4)O3)cc2c1] |
| 11 | 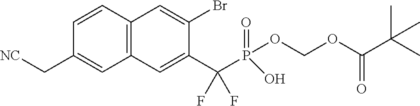 [SMILES: CC(C)(C)C(=O)OCOP(=O)(O)C(F)(F)c1cc2cc(CC#N)ccc2cc1Br] |
| 12 | 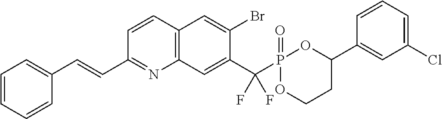 [SMILES: O=P1(C(F)(F)c2cc3nc(/C=C/c4ccccc4)ccc3cc2Br)OCCC(c2cccc(Cl)c2)O1] |
| 13 | 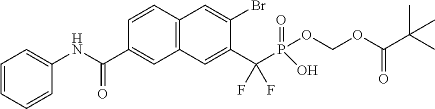 [SMILES: CC(C)(C)C(=O)OCOP(=O)(O)C(F)(F)c1cc2cc(C(=O)Nc3ccccc3)ccc2cc1Br] |
| 14 | 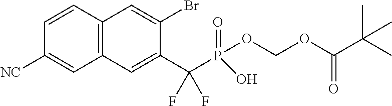 [SMILES: CC(C)(C)C(=O)OCOP(=O)(O)C(F)(F)c1cc2cc(C#N)ccc2cc1Br] |
| 15 | 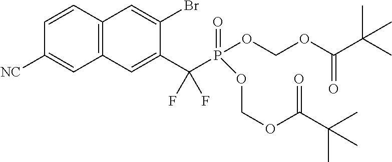 [SMILES: CC(C)(C)C(=O)OCOP(=O)(OCOC(=O)C(C)(C)C)C(F)(F)c1cc2cc(C#N)ccc2cc1Br] |

or a pharmaceutically acceptable salt thereof.

6. The method of claim 4, wherein the compound of formula (IIa) is 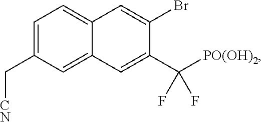
or a pharmaceutically acceptable salt thereof.

7. The method of claim 4, wherein the compound of formula (IIa) is 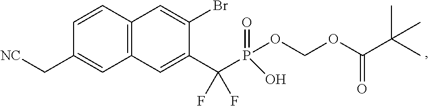 [SMILES: CC(C)(C)C(=O)OCOP(=O)(O)C(F)(F)c1cc2cc(CC#N)ccc2cc1Br]
or a pharmaceutically acceptable salt thereof.

8.-10. (canceled)

11. The method of claim 1, wherein said immune checkpoint inhibitor is an anti-PD-1 antibody, an anti-PD-L1 antibody, an anti-CTLA-4 antibody, or molecular inhibitors of these receptors.

12. The method of claim 11, wherein said anti-PD-1 antibody is selected from the group consisting of nivolumab antibody, pembrolizumab antibody, pidilizumab antibody, cemiplimab antibody, dostarlimab antibody, JTX-4014 antibody, Spartalizumab antibody, Camrelizumab antibody, Sintilimab antibody, Tislelizumab antibody, Toripalimab antibody, INCMGA00012 antibody, AMP-224 antibody, AMP-514 antibody, RMP1-14 antibody or combinations thereof.

13. The method of claim 11, wherein said anti-PDL-1 antibody is selected from the group consisting of B7-H1 antibody, BMS-936559 antibody, MPDL3280A (atezolizumab) antibody, MEDI-4736 antibody, MSB0010718C antibody, 10F-9G2 antibody, avelumab antibody, durvalumab antibody, or combinations thereof.

14. The method of claim 11, wherein said anti-CTLA-4 antibody is selected from the group consisting of ipilimumab or tremelimumab or combinations thereof.

15. The method of claim 1, wherein administration of said compound of structural Formula II is an intratumoral administration.

16. The method of claim 1, wherein administration of said compound of structural Formula II is an oral administration.

17. The method of claim 1, wherein administration of said immune checkpoint inhibitor is a systemic administration, and intratumoral administration, or a combination thereof.

18. The method of claim 1, wherein administration of said compound of structural Formula I or structural Formula II is an intratumoral administration and wherein administration of said immune checkpoint inhibitor is a systemic administration, an intratumoral administration, or a combination thereof, or
wherein administration of said compound of structural Formula I is an intratumoral administration and wherein administration of said immune checkpoint inhibitor is a systemic administration, an intratumoral administration, or a combination thereof, or
wherein administration of said compound of structural Formula I is an oral administration and wherein administration of said immune checkpoint inhibitor is a systemic administration, an intratumoral administration, or a combination thereof.

19. The method of claim 1, wherein said compound of structural Formula II is administered first, and said immune checkpoint inhibitor is administered second.

20. The method of claim 1, wherein said cancer is a solid tumor, a prostate cancer, a breast cancer, a brain cancer, a glioma, a lung cancer, a salivary cancer, a stomach cancer, a thymic epithelial cancer, a thyroid cancer, an ovarian cancer, a multiple myeloma, a leukemia, a melanoma, a lymphoma, a gastric cancer, a kidney cancer, a pancreatic cancer, a bladder cancer, a colon cancer and a liver cancer.

21.-41. (canceled)
